(19) United States
(12) Patent Application Publication
(10) Pub. No.: US 2018/0236003 A1
(43) Pub. Date: Aug. 23, 2018

(54) Cell Expansion Methods and Therapeutic Compositions

(71) Applicant: CELL IDEAS PTY LTD, Pymble, New South Wales (AU)

(72) Inventors: Balarka Banerjee, Crows Nest, New South Wales (AU); Charlotte Morgan, Copacabana, New South Wales (AU); Graham Vesey, Hornsby, New South Wales (AU); Nicolle Hannah Packer, Gordon, New South Wales (AU)

(21) Appl. No.: 15/758,005

(22) PCT Filed: Sep. 8, 2016
(86) PCT No.: PCT/AU2016/000316

§ 371 Date: Mar. 7, 2018

(30) Foreign Application Priority Data
Sep. 8, 2015 (AU) 2015903658

(51) Int. Cl. A61K 35/28; A61K 31/728; A61K 31/737; C12N 5/0775

(52) U.S. Cl. CPC A61K 35/28; C12N 5/0667; A61K 31/737; A61K 31/728

(57) ABSTRACT

The invention relates to methods for the production of mesenchymal stem cells (MSCs), in particular to methods for the large scale production of MSCs, such as allogeneic MSCs, for use in treating various diseases in humans and other animals. The invention also relates to methods which permit the selection of preferred donor cells suitable for l arge scal e production of MSCs. The invention also relates to purified MSCs prepared by the methods of the invention. The invention also relates to the use of platelet lysate in methods for preparing cultures of MSCs and to the preparation of extracellular matrix-enriched secretions. The invention also relates to methods for the preparation of compositions comprising one or more component(s) secreted from cultured MSCs, having improved stability characteristics. The invention also relates to methods for treating inflammatory conditions, including alleviating the pain thereof, by administering high molecular mass glycoconjugate-enriched conditioned media, and to methods for treating neuropathic pain by administering high molecular mass glycoconjugate-enriched conditioned media.

### FIELD

The present invention relates to methods for the production of mesenchymal stem cells (MSCs), in particular to methods for the large scale production of MSCs for use in treating various diseases in humans and other animals. In specific embodiments the methods permit efficient large scale production of allogeneic MSCs for use in therapy. The invention also relates to methods which permit the selection of preferred donor cells suitable for large scale production of MSCs. The invention also relates to purified MSCs prepared by the methods of the invention. The invention also relates to the use of platelet lysate in methods for preparing cultures of MSCs and to the preparation of extracellular matrix-enriched secretions. The present invention also relates to methods for the preparation of compositions comprising one or more component(s) secreted from cultured MSCs, such as vascular endothelial growth factor (VEGF), having improved stability characteristics. The present invention also relates to methods for treating inflammatory conditions, including alleviating the pain thereof, by administering high molecular mass glycoconjugate-enriched conditioned media. The present invention also relates to methods for treating neuropathic pain by administering high molecular mass glycoconjugate-enriched conditioned media.

### BACKGROUND

The use of allogeneic mesenchymal stem cells (MSCs) to treat various diseases in humans and animals is a rapidly expanding area of interest for many groups. At present, there are a significant number of clinical trials exploring the use of MSCs for the treatment of various diseases, including osteoarthritis, myocardial infarction, stroke, and others with clear involvement of the immune system, such as graft-versus-host disease, Crohn's disease, rheumatoid arthritis and diabetes. MSCs are being used as cell therapy to treat defects in bone and cartilage and to help in wound healing, or in combination with biomaterials in tissue engineering development.

For the commercial production of allogeneic mesenchymal stem cells it is important that the cells from a single donor can be expanded sufficiently to produce a large number of doses. Although there are known methods for the production of MSCs which are suitable for use in therapy, there are limitations in the application of such methods to the large scale production of MSCs. For example, a limitation is that there is inconsistency of proliferation potential between different tissue samples or cell samples used to establish a culture, such that one donor sample may have potential for many more cell doublings and thereby be suitable for large scale preparation of cells, whereas another donor sample may have limited potential and be unsuitable. There is currently no reliable means by which a user is able to discriminate between such samples at an early stage, with the result that cells arising from one sample may become senescent before an acceptable number of doses has been achieved, leading to wasted effort and resources.

In addition to the use of allogeneic mesenchymal stem cells (MSCs) to treat various diseases in humans and animals there is also expanding interest in the use of secretions from MSCs to treat various diseases. The secretions may take various forms, such as described in the applicant's co-pending application PCT International Publication No. WO2013/040649 entitled “Therapeutic methods and compositions”, the contents of which are incorporated herein by reference.

In embodiments described herein the invention addresses the need for improved methods of producing MSCs, and MSC-based products, in particular methods which ameliorate one or more of the limitations of known methods when applied to large scale production of MSCs, such as for the production of large numbers of doses of allogeneic cells. In embodiments the instant invention addresses the need for improved methods of producing compositions comprising MSC-secreted cytokines and growth factors, and for such compositions, the improved methods or compositions which may alleviate one or more limitations of known methods and compositions, such as ease of use, stability of compositions on storage, or therapeutic potential.

In embodiments the instant invention addresses a need for improved or alternative methods and agents for treating inflammatory conditions, including alleviating the pain thereof. In embodiments the instant invention addresses a need for improved or alternative methods and agents for treating neuropathic pain.

### SUMMARY OF INVENTION

The inventors have developed methods that enable the production of very large numbers of doses of MSCs from a single donor. The method involves firstly harvesting a large volume of adipose tissue from the donor. The lipoaspirate is then digested to isolate the stromal vascular fraction (SVF), which is then placed in to tissue culture and expanded.

As described herein the inventors have also developed innovative methods for the production of MSCs in a medium comprising platelet lysate. The inventors have identified that growth of MSCs in medium comprising platelet lysate, such as under conditions described herein, has the surprising advantage that the cells secrete high levels of high molecular mass glycoconjugates in to the tissue culture media. This allows production of high molecular mass glycoconjugate-enriched conditioned media which provides additional therapeutic advantages for the treatment of, or the alleviation of pain of, inflammatory conditions, including osteoarthritis, or for alleviation of neuropathic pain. The conditioned media generated in that manner has a higher viscosity than conditioned media generated by growth of MSCs in the absence of platelet lysate. The viscosity can thereby act as an indicator of an appropriate time to harvest cells and or conditioned media from the culture. As demonstrated herein MSCs cultured in media containing fibroblast growth factor (FGF) and epidermal growth factor (EGF) also resulted in conditioned media that was viscous.

As described herein the inventors have also developed methods for screening cells or tissue samples from different donors, or cells or tissue samples from a single donor, to select cells that are suitable for large-scale manufacturing.

In one aspect of the invention there is provided a method for treating an inflammatory condition in a subject, the method comprising administering to said subject a therapeutically effective amount of a high molecular mass glycoconjugate-enriched conditioned media.

In an embodiment the inflammatory condition is osteoarthritis. In an embodiment the method further comprises administering a therapeutically effective amount of culture-expanded MSCs. In an embodiment the method comprises administering a composition comprising culture-expanded MSCs and high molecular mass glycoconjugate-enriched conditioned media. In an embodiment the high molecular mass glycoconjugate-enriched conditioned media is prepared by culturing MSCs in media comprising platelet lysate.

In a further aspect the invention provides use of platelet lysate for the preparation of high molecular mass glycoconjugate-enriched conditioned media.

In an embodiment the platelet lysate is human platelet lysate.

In a further aspect the invention provides a method for the preparation of high molecular mass glycoconjugate-enriched conditioned media, the method comprising culturing mesenchymal stem cells (MSCs) in media comprising platelet lysate. In a further aspect the invention provides a method for the preparation of high molecular mass glycoconjugate-enriched conditioned media, the method comprising culturing mesenchymal stem cells (MSCs) in media comprising FGF and or EGF. In an embodiment the cells are cultured to greater than about 80% confluence. In an embodiment the cells are cultured for between one to ten days post-confluence. In an embodiment the cells are cultured for between one to six days post-confluence. In an embodiment the cells are cultured for about one day post-confluence, or for about two days post-confluence, or for about three days post-confluence, or for about four days post-confluence, or for about five days post-confluence, or for about six days post-confluence, or for about seven days post-confluence, or for about eight days post-confluence. In an embodiment the conditioned media has a viscosity of at least about 1.5 centistokes. In an embodiment the conditioned media has a viscosity of at least about 1.6 centistokes. In an embodiment the conditioned media has a viscosity of at least about 1.7 centistokes. In an embodiment the conditioned media has a viscosity of at least about 1.8 centistokes. In an embodiment the conditioned media has a viscosity of at least about 1.9 centistokes. In an embodiment the conditioned media has a viscosity of at least about 2 centistokes. In an embodiment the conditioned media has a viscosity of at least about 2.1 centistokes. In an embodiment the conditioned media has a viscosity of at least about 2.2 centistokes. In an embodiment the conditioned media has a viscosity of at least about 2.3 centistokes. In an embodiment the conditioned media has a viscosity of greater than 1.5 centistokes. In an embodiment the conditioned media has a viscosity of greater than 1.7 centistokes. In an embodiment the conditioned media has a viscosity of greater than 2 centistokes. In an embodiment the conditioned media has a viscosity of greater than 2.5 centistokes.

In an embodiment the MSCs are adipose tissue-derived MSCs. In an embodiment the platelet lysate is human platelet lysate. In an embodiment the media comprises about 5% to about 10% v/v platelet lysate. In an embodiment the enriched media comprises one or more of a proteoglycan, a glycosaminoglycan and a mucin. In an embodiment the enriched media comprises keratan sulphate, chondroitin sulphate or aggrecan. In an embodiment the method further comprises the step of removing the cells from the media.

In an embodiment the conditioned media, comprising secretions from the cultured cells, comprises one or more MSC-secreted growth factor(s) or cytokine(s) having improved stability compared to that MSC-secreted growth factor(s) or cytokine(s) prepared by culturing MSCs in the absence of platelet lysate. In an embodiment the conditioned media, comprising secretions from the cultured cells, comprises one or more MSC-secreted growth factor(s) or cytokine(s) having improved stability compared to that MSC-secreted growth factor(s) or cytokine(s) prepared by culturing MSCs in the absence of FGF and EGF. In an embodiment the one or more MSC-secreted growth factor(s) or cytokine(s) is selected from the group consisting of IFN-γ, IL-8, IL-9, IL-12, IL-15, TNF-α, IL-10, MCP-1, RANTES, GM-CSF, IP-10, PDGF-bb, VEGF, IL-6. In an embodiment the one or more MSC-secreted growth factor(s) or cytokine(s) is VEGF.

In an embodiment the improved stability comprises retention of at least 60% activity after one month at room temperature. In an embodiment the improved stability comprises retention of at least 70% activity after one month at room temperature. In an embodiment the improved stability comprises retention of at least 80% activity after one month at room temperature. In an embodiment the improved stability comprises retention of at least 90% activity after one month at room temperature.

In an embodiment the improved stability comprises retention of at least 60% activity after three months at room temperature. In an embodiment the improved stability comprises retention of at least 70% activity after three months at room temperature. In an embodiment the improved stability comprises retention of at least 80% activity after three months at room temperature. In an embodiment the improved stability comprises retention of at least 90% activity after three months at room temperature.

In an embodiment the improved stability comprises retention of at least 60% activity after six months at room temperature. In an embodiment the improved stability comprises retention of at least 70% activity after six months at room temperature. In an embodiment the improved stability comprises retention of at least 80% activity after six months at room temperature. In an embodiment the improved stability comprises retention of at least 90% activity after six months at room temperature.

In a further aspect the invention provides a composition comprising high molecular mass glycoconjugate-enriched conditioned media. In an embodiment the composition comprising high molecular mass glycoconjugate-enriched conditioned media comprises one or more MSC-secreted growth factor(s) or cytokine(s) having improved stability compared to that MSC-secreted growth factor(s) or cytokine(s) prepared by culturing MSCs in the absence of platelet lysate or in the absence of FGF and EGF. In an embodiment the one or more MSC-secreted growth factor(s) or cytokine(s) is selected from the group consisting of IFN-γ, IL-8, IL-9, IL-12, IL-15, TNF-α, IL-10, MCP-1, RANTES, GM-CSF, IP-10, PDGF-bb, VEGF, IL-6. In an embodiment the one or more MSC-secreted growth factor(s) or cytokine(s) is VEGF. In an embodiment the one or more MSC-secreted growth factor(s) or cytokine(s) in said composition has improved stability compared to a composition comprising the one or more MSC-secreted growth factor(s) or cytokine(s) in the absence of said high molecular mass glycoconjugate-enriched conditioned media.

In an embodiment the composition is prepared by culturing MSCs in media comprising platelet lysate. In an embodiment the composition is prepared by culturing MSCs in media comprising FGF and/or EGF.

In a further aspect the invention provides a method for preparing a composition comprising stable one or more MSC-secreted growth factor(s) or cytokine(s), wherein said stability comprises retention of at least 60% activity after one month at room temperature, the method comprising culturing mesenchymal stem cells (MSCs) in culture media comprising platelet lysate for a time sufficient to permit secretion of the one or more MSC-secreted growth factor(s) or cytokine(s) into said culture medium. In an embodiment the platelet lysate is at a concentration of 5% v/v to 10% v/v. In an embodiment the platelet lysate is at a concentration of 10% v/v. In a further aspect the invention provides a method for preparing a composition comprising stable one or more MSC-secreted growth factor(s) or cytokine(s), wherein said stability comprises retention of at least 60% activity after one month at room temperature, the method comprising culturing mesenchymal stem cells (MSCs) in culture media comprising FGF and/or EGF for a time sufficient to permit secretion of the one or more MSC-secreted growth factor(s) or cytokine(s) into said culture medium. In an embodiment the FGF and/or the EGF is at a concentration of between 10 ng/ml and 30 ng/ml. In an embodiment the FGF is at a concentration of 20 ng/ml. In an embodiment the EGF is at a concentration of 20 ng/ml. In an embodiment the one or more MSC-secreted growth factor(s) or cytokine(s) is selected from the group consisting of IFN-γ, IL-8, IL-9, IL-12, IL-15, TNF-α, IL-10, MCP-1, RANTES, GM-CSF, IP-10, PDGF-bb, VEGF, IL-6. In an embodiment the one or more MSC-secreted growth factor(s) or cytokine(s) is VEGF. In an embodiment the MSCs are human adipose derived MSCs.

In an embodiment said method comprises culturing the MSCs in media comprising platelet lysate to a cell density of greater than about 80% confluence. In an embodiment said method comprises culturing the MSCs in media comprising platelet lysate to a cell density of greater than about 85% confluence. In an embodiment said method comprises culturing the MSCs in media comprising platelet lysate to a cell density of greater than about 90% confluence. In an embodiment said method comprises culturing the MSCs in media comprising platelet lysate to a cell density of greater than about 95% confluence. In an embodiment said method comprises culturing the MSCs in media comprising platelet lysate to a cell density of greater than about 100% confluence. In an embodiment said culturing is for a time sufficient to permit preparation of a conditioned media having a viscosity of at least about 1.5 centistokes (cSt). In an embodiment said culturing is for a time sufficient to permit preparation of a conditioned media having a viscosity of at least about 1.6 centistokes. In an embodiment said culturing is for a time sufficient to permit preparation of a conditioned media having a viscosity of at least about 1.7 centistokes.

In an embodiment the method further comprises collecting conditioned media from said culture. In an embodiment the method further comprises collecting conditioned media and culture-expanded MSCs from said culture.

In embodiments said stability comprises, optionally, retention of at least 70% activity of said one or more MSC-secreted growth factor(s) or cytokine(s) after one month at room temperature, retention of at least 80% activity of said one or more MSC-secreted growth factor(s) or cytokine(s) after one month at room temperature, retention of at least 90% activity of said one or more MSC-secreted growth factor(s) or cytokine(s) after one month at room temperature, retention of at least 70% activity of said one or more MSC-secreted growth factor(s) or cytokine(s) after three months at room temperature, retention of at least 80% activity of said one or more MSC-secreted growth factor(s) or cytokine(s) after three months at room temperature, retention of at least 90% activity of said one or more MSC-secreted growth factor(s) or cytokine(s) after three months at room temperature, retention of at least 60% activity of said one or more MSC-secreted growth factor(s) or cytokine(s) after six months at room temperature, retention of at least 70% activity of said one or more MSC-secreted growth factor(s) or cytokine(s) after six months at room temperature, retention of at least 80% activity of said one or more MSC-secreted growth factor(s) or cytokine(s) after six months at room temperature, or retention of at least 90% of said one or more MSC-secreted growth factor(s) or cytokine(s) activity after six months at room temperature.

In a further aspect the invention provides a composition comprising culture-expanded MSCs and high molecular mass glycoconjugate-enriched conditioned media.

In an embodiment a composition according to the invention is a pharmaceutical composition. In an embodiment the pharmaceutical composition is an injectable composition. In an embodiment the pharmaceutical composition is a composition for topical application, such as to the skin, gum, or mucous membrane of a subject. In an embodiment the pharmaceutical composition is a cream, gel, liquid or lotion. In an embodiment the pharmaceutical composition comprises high molecular mass glycoconjugate-enriched conditioned media formulated in a gel or cream for topical application. In an embodiment the composition does not comprise a surfactant.

In an embodiment of the composition comprising culture-expanded MSCs and high molecular mass glycoconjugate-enriched conditioned media the cells are not adhered in a container containing said composition.

In a further aspect the invention provides a pharmaceutical composition comprising (i) high molecular mass glycoconjugate-enriched conditioned media, or (ii) culture-expanded MSCs and high molecular mass glycoconjugate-enriched conditioned media, and a pharmaceutically acceptable carrier, excipient or adjuvant.

In a further aspect the invention provides a method of screening a sample of MSCs for suitability for use in large scale production of cultured MSCs, the method comprising the steps of (i) culturing cells of said sample in a culture medium comprising platelet lysate or FCS for 1, 2, or 3 passages and (ii) culturing said cells or an aliquot thereof after step (i) in a culture medium comprising allogeneic serum, wherein continued proliferation of cells in said culture medium comprising allogeneic serum is indicative of a sample suitable for use in the production of large scale numbers of cultured MSCs.

In an embodiment suitability for use in large scale production of cultured MSCs comprises the capacity for at least 25 population doublings before senescence. In an embodiment suitability for use in large scale production of cultured MSCs comprises the capacity for at least 30 population doublings before senescence. In an embodiment suitability for use in large scale production of cultured MSCs comprises the capacity for at least 35 population doublings before senescence. In an embodiment suitability for use in large scale production of cultured MSCs comprises the capacity for at least 40 population doublings before senescence. In an embodiment suitability for use in large scale production of cultured MSCs comprises the capacity for at least 45 population doublings before senescence.

In an embodiment the sample of MSCs is an adipose tissue-derived sample of MSCs. In an embodiment the sample of MSCs is selected from human, canine, equine and feline MSCs.

In a further aspect the invention provides a method of screening a sample of MSCs for unsuitability for use in large scale production of cultured MSCs, the method comprising the steps of (i) culturing cells of said sample in a culture medium comprising platelet lysate or FCS for 1, 2, or 3 passages and (ii) culturing said cells or a portion thereof from step (i) in a culture medium comprising allogeneic serum, wherein failure of the cells to proliferate in said culture medium comprising allogeneic serum is indicative of a sample unsuitable for use in the production of large scale numbers of cultured MSCs.

In an embodiment failure of the cells to reach confluence is indicative of failure of the cells to proliferate.

In a further aspect the invention provides a method for large scale production of cultured MSCs, the method comprising the steps of (i) obtaining a cell sample or tissue sample comprising MSCs, (ii) culturing at least a portion of said sample in a culture medium comprising platelet lysate or FCS for 1, 2, or 3 passages to provide a cultured cell population, (iii) culturing a portion of said cultured cell population from step (ii) in a culture medium comprising allogeneic serum, wherein upon continued proliferation of cells in said culture medium comprising allogeneic serum (iv) culturing at least a portion of said cultured cell population from step (i) or step (ii) in a culture medium comprising platelet lysate or FCS for additional passages to provide a large scale preparation of cultured MSCs.

In an embodiment the cell sample is a cell suspension comprising stromal vascular fraction of adipose tissue. In an embodiment the cell sample comprises isolated MSCs. In an embodiment the cell sample comprises culture expanded MSCs. In an embodiment the cells of at least one of steps (i) to (iv) are cells that have been frozen. In an embodiment step (iv) comprises culturing said cell population for 10 or more additional population doublings; or for 15 or more additional population doublings; or for 20 or more additional population doublings; or for 25 or more additional population doublings; or for 30 or more additional population doublings; or for 35 or more additional population doublings. In an embodiment the method further comprises harvesting culture expanded MSCs after said additional passages and, optionally, aliquoting said harvested cells into individual containers constituting a therapeutic dose of MSCs. In an embodiment a therapeutic dose of MSCs comprises between about 2 million and about 10 million MSCs. In an embodiment a therapeutic dose of MSCs comprises about 5 million MSCs.

In a further aspect of the invention there is provided a method for the preparation of a purified population of culture-expanded adipose tissue-derived mesenchymal stem cells, the method comprising steps of:
(i) obtaining a sample of adipose tissue from a subject,
(ii) incubating said adipose tissue under suitable conditions to at least partially digest said adipose tissue, wherein said conditions comprise incubation in a buffer comprising calcium at a concentration of between 50 mg/L to 500 mg/L and in the presence of collagenase at a concentration of between 0.2% wt/vol to 0.02% wt/vol,
(iii) centrifuging said incubated adipose tissue to obtain stromal vascular fraction (SVF) cells,
(iv) seeding said SVF cells into tissue culture at a desired seeding density,
(v) incubating said culture under appropriate conditions in serum-supplemented media to at least 85% flask confluence,
(vi) harvesting MSCs from said culture,
(vii) passaging said harvested MSCs or progeny thereof for at least 10 population doublings to obtain culture-expanded MSCs.

In an embodiment the collagenase concentration is 0.05% wt/vol. In an embodiment the calcium is at a concentration between 300 mg/L to 400 mg/L. In an embodiment the calcium is at a concentration of 330 mg/L. In an embodiment said conditions comprise mixing on an orbital rotor. In an embodiment the seeding density is between about 5,000 and about 15,000 cells per cm² of tissue culture flask. In an embodiment the tissue culture comprises culturing said cells on microcarrier beads or discs. In an embodiment the serum-supplemented media comprises FCS or platelet lysate. In an embodiment the FCS is at a concentration of between about 5% and 10%. In an embodiment the platelet lysate is at a concentration of between about 5% and 10%. In an embodiment the platelet lysate is at a concentration of 10% v/v. In an embodiment the method further comprises, prior to the cells having undergone more than three passages, an aliquot of said cells is cultured in a culture medium comprising allogeneic serum to identify cells suitable for continued use in the production of large scale numbers of cultured MSCs. In an embodiment the method further comprises continued passaging, in media comprising FCS or platelet lysate, of cells identified as suitable for continued use in the production of large scale numbers of cultured MSCs. In an embodiment the method comprises passaging harvested MSCs or progeny thereof for at least 25 population doublings; or for at least 30 population doublings; or for at least 35 population doublings; or for at least 40 population doublings; or for at least 45 population doublings, to obtain culture-expanded MSCs.

### Abbreviations

DMEM Dulbecco's Modified Eagles Medium.

SVCs stromal vascular cells.

SVF stromal vascular fraction.

MSC mesenchymal stem cell(s).

FCS fetal calf serum (may also be abbreviated herein to FBS, being fetal bovine serum).

ABC ammonium bicarbonate buffer.

wt/vol weight/volume.

(v/v) or v/v volume/volume.

VEGF vascular endothelial growth factor.

(cSt) centistokes

### BRIEF DESCRIPTION OF DRAWINGS

FIG. 1. Comparison of the viscosity of culture media of MSCs grown in fetal calf serum and in platelet lysate. MSCs were cultured in DMEM supplemented with either 10% FCS (squares) or 10% platelet lysate (circles) and the viscosity of the respective medium determined after 0, 1, 3, and 6 days post confluence. Kinematic viscosity was determined as described in Example 5 and is stated in centistokes (cSt).

FIG. 2. Stability of VEGF. The amount of VEGF in supernatant harvested from culture of human adipose-derived MSCs cultured in DMEM plus 5% platelet lysate for 10 days was measured using an ELISA, with assays being conducted at various times over six months storage of the supernatant composition at room temperature (approximately 23° C.).

FIG. 3. Comparison of the percentage change in 14 different cytokines in viscous (light shading) and non-viscous (dark shading) conditioned media after storage at 22° C. for 5 months. Values are stated as percent change to starting material stored at −80° C.

FIG. 4. Comparison of the viscosity of conditioned media from cultures that are less than or greater than 80% confluent.

### DESCRIPTION OF EMBODIMENTS

Throughout this specification, reference to “a” or “one” element does not exclude the plural, unless context determines otherwise. Similarly, reference to “an embodiment” does not exclude the characteristic of that described embodiment applying in combination with one or more other embodiments described, unless the context determines otherwise.

In the context of this specification, the term “comprising” means including, but not necessarily solely including. Furthermore, variations of the word “comprising”, such as “comprise” and “comprises”, have correspondingly varied meanings. Hence, the term “comprising” and variations thereof is used in an inclusive rather than exclusive meaning such that additional integers or features may optionally be present in a composition, method, etc. that is described as comprising integer A, or comprising integer A and B, etc.

In the context of this specification the terms “about” and “approximately” will be understood as indicating the usual tolerances that a skilled addressee would associate with the given value.

In the context of this specification, where a range is stated for a parameter it will be understood that the parameter includes all values within the stated range, inclusive of the stated endpoints of the range. For example, a range of “5 to 10” will be understood to include the values 5, 6, 7, 8, 9, and 10 as well as any sub-range within the stated range, such as to include the sub-range of 6 to 10, 7 to 10, 6 to 9, 7 to 9, etc., and inclusive of any value and range between the integers which is reasonable in the context of the range stated, such as 5.5, 6.5, 7.5, 5.5 to 8.5 and 6.5 to 9, etc.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of this application.

In the context of this specification, the terms “plurality” and “multiple” mean any number greater than one.

It is to be noted that reference herein to use of the inventive methods and compositions in treatment or therapy, or in the obtaining of tissue or cells from donors, will be understood to be applicable to human and non-human, such as veterinary, applications. Hence it will be understood that, except where otherwise indicated, reference to a donor, patient, subject or individual means a human or a non-human, such as an individual of any species of social, economic, agricultural or research importance including but not limited to members of the classifications of ovine, bovine, equine, porcine, feline, canine, primates, rodents, especially domesticated or farmed members of those classifications, such as sheep, cattle, horses, pigs, cats and dogs.

Where examples of various embodiments or aspects of the invention are described herein they will generally be prefaced by appropriate terms including “such as” or “for example”, or “including”. It will be understood that the examples are being described as inclusive possibilities, such as for the purpose of illustration or understanding and are not, unless the context indicates otherwise, being provided as limiting.

The pharmaceutical composition referred to herein may also be referred to as a medicament, such as when intended for therapeutic use. Hence, it will be understood that where the invention is described as including the use of a composition of described components for the preparation of a pharmaceutical composition for an intended therapeutic purpose, that description equally means use for the preparation of a medicament for that intended therapeutic purpose, unless the context indicates otherwise.

In the context of this specification and in particular in the context of the use of culture-expanded MSCs for therapeutic use, a “dose” is an aliquot of culture-expanded MSCs in sufficient numbers which may be administered to an individual for the purposes of providing a therapeutic benefit to the individual. Depending on the context, reference herein to a “dose” may or may not include additional therapeutic components, such as high molecular mass glycoconjugate-enriched conditioned media. The actual number of cells in the “dose” is typically in the range of about 2 million to about 20 million cells, preferably such as about 4 million to about 10 million cells, or preferably about 5 million cells. It will be understood that a “dose” may be administered in one or more than one injections. A “dose” may also be seen as a unit of suitable storage of culture-expanded MSCs.

The terms “treating”, “treatment”, “therapy” and the like in the context of the present specification refer to the alleviation of the symptoms and/or the underlying cause of the condition or disease, such as inflammatory disorder. In certain embodiments a treatment will slow, delay or halt the progression of a disorder or the symptoms of the disorder or injury, or reverse the progression of the disorder or injury, at least temporarily. Hence, in the context of this invention the word “treatment” or derivations thereof such as “treating” when used in relation to a therapeutic application includes all aspects of a therapy, such as the alleviation of pain associated with the condition being treated, alleviation of the severity of the condition being treated, improvement in one or more symptoms of the condition being treated, etc. Use of the word “treatment” or derivatives thereof will be understood to mean that the subject being “treated” may experience any one or more of the aforementioned benefits.

The term “preventing” and the like, in the context of the “prevention” of disease, refers to hindrance of the progression of the symptoms or the underlying cause of the disease. It will be understood that complete prevention of a disease may occur, such that the disease does not occur in a treated animal or subject. Equally, it will be understood that the term includes partial prevention, such as the failure of a disease to progress to the typical state observed in an animal or subject left untreated.

It will be understood that where the description herein indicates a potential advantage of or potential improvement offered by an embodiment of the invention, not all embodiments will be required to meet that advantage or improvement.

To the extent that it is permitted, all references cited herein are incorporated by reference in their entirety.

In embodiments described herein the invention relates to improved methods for the large scale preparation of culture expanded mesenchymal stem cells (MSCs), such as for use in therapeutic methods. The methods allow the production of millions of therapeutic doses of MSCs from a single donor preparation of adipose tissue.

Mesenchymal stem cells (MSCs) are post-natal, multipotent, adult stem cells. Mesenchymal stem cells (MSCs) are present in many tissues in the body and play an important role in tissue repair and regeneration. For therapeutic purposes MSCs are commonly harvested from bone marrow, placenta, cord blood and adipose tissue. In many circumstances the cells are expanded by tissue culture prior to use. The instant invention provides improved methods for the large scale production of culture expanded cells, including methods for the selection of donor cells that have preferred potential for use in such large scale production.

### Adipose Tissue

In the context of this invention, the mesenchymal stem cells (MSCs) preferably originate from adipose tissue. By “originate” is meant the tissue type that the MSCs are isolated from for use in the methods or compositions of the present invention. The MSCs may be isolated from a tissue specifically for the purposes of the methods and compositions of the invention, or the MSCs may have previously been isolated from a tissue source in a procedure unrelated to the methods or compositions of the invention.

Adipose tissue may be human adipose tissue or mammalian animal adipose tissue, such as canine, equine, or feline. Typically the source of the adipose tissue will be of the same species as the intended recipient of the MSCs. The adipose tissue may comprise “white” adipose tissue, or “brown” adipose tissue.

Along with an abundance of MSCs, adipose tissue also comprises immune cells, vascular smooth muscle cells, endothelial cells, and pericytes, which together with the MSCs collectively are termed the stromal vascular fraction (SVF).

### Collection and Processing of Adipose Tissue

Adipose tissue is collected from donors by liposuction or by excision. In the methods of the invention directed to large scale manufacture of cultured MSCs a large amount (about 200 gams to 2000 grams) of lipoaspirate is collected, or made available, as starting material, as this will influence the number of cells that can be manufactured.

To produce cells from dogs, adipose tissue is typically collected by excision from the falciform, the inguinal fat pads or the shoulder. To produce cells from horses, adipose tissue is typically collected by excision from the tail-base, the chest or the abdomen. To produce cells from humans, adipose tissue is collected from the abdomen, thighs or buttocks.

The adipose tissue may initially be processed by mechanically dissociating adipose tissue using techniques which are readily available in the art. Any suitable method for the mechanical dissociation of adipose tissue may be used, for example by mincing adipose tissue with blades, or with scissors, or by forcing adipose tissue through screens or meshes with a pore size sufficient to break the tissue into isolated cells or small pieces of adipose tissue, or a combination of these techniques. In preferred methods, where mechanical dissociation is used, the adipose tissue is finely minced with scissors prior to digestion.

It has surprisingly been discovered that the number of cells extracted from the adipose tissue can be maximised whilst keeping the cells viable, hence maximising SVF cell yield and viability, through the use of an optimal adipose tissue digestion, such as described in the following paragraphs. Where maximising SVF cell yield and viability is of importance to the operator, such methods will be preferred.

Adipose tissue is digested by incubation with collagenase in the presence of an appropriate buffer. In the digestion collagenase is added to a final concentration of between about 0.2% weight/volume (wt/vol) and about 0.02% wt/vol, such as about 0.2% wt/vol, or about 0.15% wt/vol, or about 0.1% wt/vol, or about 0.9% wt/vol, or about 0.08% wt/vol, or about 0.07% wt/vol, or about 0.06% wt/vol, or about 0.05% wt/vol, or about 0.04% wt/vol, or about 0.03% wt/vol, or about 0.02% wt/vol. In a preferred embodiment the collagenase in the digestion step is added to a final concentration of about 0.05% wt/vol.

The inventors have identified that improved cell yield and viability is also aided by the use of a calcium containing buffer during the digestion. The concentration of calcium during the digestion step is in the range of about 50 mg/L to about 500 mg/L, such as about 50 mg/L, 100 mg/L, 125 mg/L, 150 mg/L, 175 mg/L, 200 mg/L, 225 mg/L, 250 mg/L, 300 mg/L, 325 mg/L, 330 mg/L, 350 mg/L, 375 mg/L, 400 mg/L, 425 mg/L, 450 mg/L, 475 mg/L, or 500 mg/L. In a preferred embodiment the concentration of calcium in the digestion is about 330 mg/L. In a preferred embodiment the buffer is calcium containing Ringers buffer or calcium containing phosphate buffered saline (PBS).

The adipose tissue is incubated in the presence of the collagenase at an appropriate temperature, such as about 37° C., for an appropriate time, such as about 30 to about 120 minutes. In a preferred embodiment the incubation is for about 90 minutes. The inventors have identified that improved cell yield and viability is also aided by careful mixing during the digestion step to release cells, as too much mixing, or too rigorous mixing, will affect cell viability. Mixing may be conducted by hand, such as by gentle inversion of the material at intervals during the incubation, for example about every 15 minutes. Mixing may be conducted by mechanical means, such as mixing in the range of about 50 rpm to about 200 rpm, for example on an orbital rotator. In a preferred embodiment the mixing is either by hand or is at about 100 rpm on an orbital rotator.

The preparation of an adipose tissue-derived cell suspension may comprise a centrifugation step. The centrifugation of isolated cells or small aggregates or pieces of adipose tissue suspended in a liquid, such as a medium, is at approximately 500 g for 10 minutes, or for sufficient time and at a sufficient g-force to generate a cell pellet which comprises adipose-derived non-adipocyte cells, above which is a layer of medium, floating above which in turn is a layer which comprises the viable adipocytes, and floating at the top is a layer of lipid which is derived from ruptured adipocytes. Following centrifugation, the lipid layer, the adipocytes and the medium layer will preferably be discarded and the pelleted material, referred to as the SVF and comprising MSCs, is retained. The pelleted cells may also be referred to as adipose-derived non-adipocyte cells or as SVF cells.

### Tissue Culture

The SVF cells are placed in to tissue culture. The SVF contains a mixed population of cell types, including MSCs. Red blood cells (RBCs) are typically also present in the SVF, as may be cell clusters. The RBCs are typically present as a contaminant at low numbers, the majority of the RBCs having been removed by washing the adipose tissue pieces prior to digestion with collagenase. Other researchers typically purify the MSCs from the other cell types prior to performing tissue culture, such as by fluorescence activated cell sorting (FACS) or immune-magnetic cell separation (IMS) or partially purified by density gradient centrifugation, or the lysis or removal of red blood cells, or removal of cell clusters. In the methods of the invention there is no need for further purification of the MSCs from the SVF prior to tissue culture. The inventors have surprisingly identified that prior purification of the MSCs is not required and in fact that improved MSC yield and viability on culturing is obtained when the MSCs are not further purified from the SVF prior to culturing. Without wishing to be bound by theory, with respect to the step of removing red blood cells by lysis as conducted in some known methods, the inventors suspect that lysing red blood cells negatively affects MSC yield and viability.

The SVF cells are seeded at a level of between about 5,000 and about 15,000 cells per cm² of tissue culture flask. The MSCs are the most rapidly growing cells present in the SVF and out-grow the other cell types resulting in a pure population of MSCs. Surprisingly, despite the presence of the competing contaminant or non-MSC cells, this approach to expansion gives higher cell yields than purifying a starting population of MSCs prior to tissue culture.

Methods for culturing MSCs are known in the art and include, for example, culturing the cells to form an adherent cell culture, such as a confluent adherent cell culture. At any appropriate time during, or after, culturing of the cells supernatant is harvested, such as from an adherent cell culture, which may be a confluent adherent cell culture, and optionally, removing cells from said supernatant to form a composition comprising adipose tissue-derived secretions. Any appropriate medium may be used for culture of the cells. Appropriate media include, for example, DMEM, RPMI and minimal essential media. In an embodiment the cells are cultured in DMEM.

The cell culture is preferably in the presence of sterile serum. The concentration of the serum in the culture may be any suitable concentration which assists culturing of adipose tissue-derived cells, such as for example in the range of about 1% volume/volume (v/v) to about 30% v/v, such as about 10% v/v, or about 15% v/v or about 20% v/v. The serum may be any appropriate serum for the culturing of adipose tissue-derived cells, such as a commercial fetal calf serum, or a serum prepared in house, such as by methods known in the art. In embodiments of the invention the serum is autologous, having been prepared from the same individual from which the adipose tissue was obtained, or allogeneic. Typically, the cells are cultured at 37° C. with 5% CO₂.

In embodiments of the invention the serum is from a different species. For example, cells may be canine, equine or human and may be cultured in media containing a serum which is other than canine, equine or human, respectively. In embodiments of the invention the cells are cultured in media supplemented with fetal calf serum. In embodiments of the invention the media comprises 10% FCS. Cells may be switched from FCS to allogeneic serum at a later passage, such as the final passage to remove the FCS from the final product.

In methods for the large scale preparation of cultured MSCs, the cells are typically grown to at least 85% flask confluence (method of confluence determination is via a microscope) in culture media and serum for 4-21 days in a CO₂ incubator at 37° C.

The invention also provides for MSCs cultured on microcarriers, for example in a stirred bioreactor. The MSCs can be cultured on microcarrier beads (90-400 um) which may be uncoated, or coated with particular proteins such as collagen and may be treated to have specific charge distribution on the surface. The microcarriers may be used for the growth of cells in a number of vessels such as spinner flask, microbioreactors, stirred tank, rocking platforms or microgravity. Cells may also be cultured on Fibra-Cel® discs (New Brunswick Scientific, Edison, N.J.), which may be maintained in a bioreactor as a suspended stirred system or as a static packed bed.

The cells in culture, such as MSCs, secrete components into the culture medium such that the liquid phase obtained from culture will contain components secreted from the cells. These secreted components may collectively be referred to herein as the secretome. The liquid phase generated during the cell culturing may be referred to herein as secretions or, where the cells in culture are adipose tissue-derived non-adipocyte cells, as secretions from adipose tissue-derived non-adipocyte cells. Alternatively the liquid phase generated during culturing and optionally obtained from culturing may also be referred to herein as conditioned media.

### MSCs Cultured in Platelet Lysate and Production of High Molecular Mass Glycoconjugate-Enriched Media

In embodiments of the invention the cells are cultured in platelet lysate. In the methods described herein platelet lysate is preferred as the serum source for tissue culture of human cells. Platelet lysate is used at a concentration of about 1% volume/volume (v/v) to about 20% v/v, such as about 1%, or about 2%, or about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, preferably at a concentration in the range of about 2% to about 7.5%, such as about 5% v/v, or more preferably at a concentration range of about 7.5% to 15%, such as about 10%.

Platelet lysate gives a high cell yield of cells and by the growth of the cells in platelet lysate-supplemented media the inventors have surprisingly enabled the production of conditioned medium that contains high levels of extracellular matrix (ECM) components. As demonstrated herein cells grown in platelet lysate secrete high concentrations of extracellular matrix (ECM) components, which may include proteoglycans, glycosaminoglycans and mucins.

As described in the examples herein the inventors have also shown that a similarly very viscous conditioned media can be achieved by growing the cells in media containing epidermal growth factor (EGF) and/or basic fibroblast growth factor (FGF). The concentration of EGF in the culture media may be in the range of about 10 ng/ml to about 30 ng/ml, such as about 20 ng/ml. The concentration of FGF in the culture media may be in the range of about 10 ng/ml to about 30 ng/ml, such as about 20 ng/ml.

The enriched conditioned media will generally be referred to herein as high molecular mass glycoconjugate-enriched media or conditioned media comprising ECM components. Preliminary results of kinematic viscosity supported the deduction that the viscosity component is a GAG, a proteoglycan, aggrecan, versican, lumican, mucin or other high molecular weight molecule or complex of molecules.

The invention thus also provides methods for production of high molecular mass glycoconjugate-enriched media. This media provides additional therapeutic advantages for the treatment of inflammatory conditions, including osteoarthritis. It is relevant to note that although the production of high molecular mass glycoconjugate-enriched media by culturing MSCs in platelet lysate-supplemented media was initially developed by the inventors using MSCs obtained from adipose tissue by the methods of the invention described herein, having made that invention, the inventors consider that the MSCs for use in preparing the enriched media are not limited only to those obtained from adipose tissue by the collection and digestion methods described herein.

For example, it is described herein that in the preparation of an adipose tissue-derived SVF for tissue culture of large scale MSC production, the SVF is preferably not further fractionated or purified to isolate MSCs prior to initial tissue culture steps. In the methods of preparing high molecular mass glycoconjugate-enriched media by culturing MSCs in platelet lysate-supplemented media the MSCs may be prepared according to that method or they may be prepared or obtained according to any other suitable method which provides MSCs that may be used in tissue culture.

For example, whereas the preferred large scale method does not purify MSCs from the SVF prior to initial culture, MSCs for use in the methods of preparing high molecular mass glycoconjugate-enriched media by culturing MSCs in platelet lysate-supplemented media may be further purified by any suitable means after initial isolation of the SVF and before placing in media comprising platelet lysate.

As a further example, the MSCs for use in the methods of preparing high molecular mass glycoconjugate-enriched media may be purified and stored MSCs, such as cells having previously been purified and or culture expanded by cell culture, or may be cells retrieved from frozen storage.

It will be also noted that the MSCs for use in the methods of preparing high molecular mass glycoconjugate-enriched media are not limited to adipose tissue-derived MSCs. MSCs for use in the methods of preparing high molecular mass glycoconjugate-enriched media may therefore be sourced from any appropriate source of MSCs, for example from bone marrow, dental pulp, adipose tissue, chord tissue, chord blood and circulating blood.

Furthermore, the invention thus also provides a combination MSCs and conditioned media comprising extracellular matrix (ECM) components, such media may also be referred to herein as high molecular mass glycoconjugate-enriched media. The inventors envisage that a pharmaceutical composition of such a combination of cells and conditioned media comprising ECM may provide additional therapeutic advantages over the use of MSCs alone or conditioned media from culture of MSCs alone. The methods of the invention thus allow for a composition comprising MSCs and conditioned media comprising ECM components, in which the conditioned media comprising ECM components may be that in which the cells of the composition were expanded or may be that from expansion of MSCs not comprised in the composition. The use of a composition comprising MSCs and conditioned medium comprising ECM components in which those MSCs were expanded may provide additional advantages. The MSCs or the high molecular mass glycoconjugate-enriched media or a combination thereof may be in the form of a pharmaceutical composition. In such a form the composition may typically include a pharmaceutically acceptable carrier, excipient or adjuvant or at least will typically not include a component which is incompatible with therapeutic use in an intended subject. The pharmaceutical composition may be in a form for use by injection or in a form for use by topical application. Where the composition comprises MSCs the composition will typically be in a form suitable for injection. Where the composition is for topical application the composition will typically comprise a gel, cream, liquid or lotion formulation.

The platelet lysate may be obtained from any appropriate source. A suitable commercial source is PLT Max from Mill Creek Life Sciences (Rochester, Minn., USA). The platelet lysate may be derived from the same or a different species as the MSCs being cultured. In preferred embodiments the platelet lysate is derived from the same species as the MSCs being cultured. In this context, platelet lysate “derived from” describes platelet lysate that has been prepared from a blood sample, such as by isolation of platelets from the blood sample followed by lysing of the isolated platelets. The blood sample from which the platelet lysate is derived may or may not be from the same individual as the MSCs, or where MSCs are prepared from adipose tissue, the adipose tissue for preparation of the MSCs. Typically, the blood sample is from a different individual from that from whom the MSCs or the adipose tissue is or was obtained.

The platelet lysate may be prepared from fresh whole blood or from stored whole blood using methods or kits known to the skilled addressee. The platelet lysate may be from a single donor or may be from pooled blood or cells. The platelet lysate may be prepared from expired transfusable whole blood or platelets, such as about 5 to 7 days post-collection. The platelet lysate may be prepared from blood using a commercially available kit such as the platelet lysate kit from MacoPharma (France). In an embodiment the platelet lysate is prepared from blood collected in the presence of an anticoagulant, such as citrate. The blood is centrifuged under appropriate conditions, such as at 200 g for about 20 minutes, followed by collection of the platelets (top layer) which are then subjected to freeze-thawing to lyse the cells. Typically, multiple rounds of freeze-thawing are conducted, such as two, three, four, or more rounds. The lysed platelets are centrifuged to allow the pelleted cell fragments to be discarded, for example at 4000 g for about 10 minutes. The platelet lysate may be sterilised, such as by filtering through a suitable matrix, such as a 0.22 micron filter, and stored under appropriate conditions, such as −80 C, until use.

In the methods of the invention, when cells are cultured in platelet lysate, heparin is typically also added to the cell culture media to prevent clotting. Heparin may be included in the cell culture media at a range of about 0.6 IU/mL to about 5 IU/mL. For example heparin may be included at a concentration of about 0.6 IU/mL, about 0.8 IU/mL, about 1 IU/mL, about 1.5 IU/mL, about 2 IU/mL, about 2.5 IU/mL, about 3 IU/mL, about 2.5 IU/mL, about 4 IU/mL, about 4.5 IU/mL, or about 5 IU/mL. In preferred embodiments heparin is included at about 2 IU/mL.

As described in the Examples herein the enriched conditioned media was demonstrated to have a higher viscosity than conditioned media generated by culturing MSCs in media supplemented with FCS (in the absence of platelet lysate, FGF and EGF as exemplified herein). The viscosity was shown to increase with increasing cell density of the MSCs. In embodiments of the invention, the enriched conditioned media has a viscosity of at least about 1.5 centistokes, or at least about 1.6 centistokes, or at least about 1.7 centistokes.

The skilled addressee will appreciate that the viscosity may be measured by an alternative means, such as by measuring dynamic viscosity rather than by measuring kinematic viscosity as was done in the Examples herein. It will therefore be understood that in embodiments of the invention where viscosity is a descriptive feature of the invention, a composition which includes the described features of the invention herein but which is said to have a viscosity described in alternative units, for example in centipoise (cP), as may occur if the viscosity was measured by dynamic viscosity, will still lie within the scope of this invention if the viscosity, when measured or described in units of centistokes, is at least about 1.5 centistokes, or at least about 1.6 centistokes, or at least about 1.7 centistokes, or other value as defined herein.

As demonstrated in the Examples herein the enriched conditioned media of the invention provides for improved stability of secreted cell components, exemplified by vascular endothelial growth factor (VEGF) and multiple other cytokines and growth factors. In the Examples the improved stability is characterised by determining the stability of VEGF and other cytokines and growth factors during storage at room temperature over an extended period of time. In contrast to the expectation from the literature which teaches the half-life of secretions such as VEGF at room temperature to be hours, storage of the enriched conditioned media at room temperature (which was approximately 23° C.) for a period of six months did not result in appreciable loss of VEGF, as measured using an ELISA. The results also show that multiple different cytokines and growth factors were more stable in the viscous material than in the non-viscous when stored at room temperature over a period of five months.

### Screening of Donors

The inventors have identified that the number of doses of culture-expanded MSCs that can be obtained by expanding cells from a single donor varies greatly between cells from different donors. The cells from some donors stop growing after a limited number of cell doublings whereas cells from other donors continue to grow to a greater number of cell doublings. The inventors have also identified that there may be variation in the number of doses that can be obtained by expanding cells obtained from different locations of a given individual donor animal. The reasons for these differences are not understood and there are no satisfactory methods currently available to analyse cells at a relatively early stage to predict how many doses can be produced from the expansion of particular cells, being cells obtained from a particular individual donor or a particular location on a donor. This means that cells from different donors or locations have to be grown and expanded and then thrown away if they do not reach the desired number of cell doublings. As a result, the absence of a suitable predictive method can lead to increased expense and increased time required for the large scale preparation of MSCs, such as for therapeutic purposes.

The inventors have developed and herein describe methods for screening cells from different donors or locations to rapidly identify which donor's cells will be suitable for manufacturing large numbers of cells.

The inventors have developed and herein describe methods for screening cells from different donors or locations to rapidly identify which donor's cells will be suitable for manufacturing large numbers of cells, which method involves changing the tissue culture medium in which the cells are being cultured from platelet lysate or FBS-supplemented medium to allogeneic serum-supplemented medium. As a short-hand terminology this method of screening may herein be referred to as a ‘serum switch’ method. Cells from donors that are not suitable for the production of large numbers of cells do not cope well with this change in serum. They stop replicating and fail to reach confluence.

In the methods of screening based on changing the tissue culture medium, typically cells are cultured in platelet lysate or FBS for one, two, or three passages, preferably for two passages, and then the platelet lysate or FBS is changed for allogeneic serum. The concentration of allogeneic serum used in this aspect of the invention is typically about 10% v/v to about 20% v/v. Cells that are suitable for large-scale manufacturing will continue to grow and reach confluence in the allogeneic serum. The majority of donor cell samples do not continue to grow in the allogeneic serum, in that they fail to reach confluence.

In an embodiment of this method of screening, a portion of the cells from a donor sample is taken at an early passage, for example after two passages, as a “test” sample, which may also be referred to as a sacrificial test sample as it and its progeny may be discarded after testing. The test sample of cells is subjected to the described ‘serum switch’. If the cells cope with the change to allogeneic serum-supplemented media, in that they continue to grow and reach confluence, then the user returns to the sample from which the ‘test’ cells were drawn and continues with the large scale culturing of those cells or a portion thereof in the preferred growth medium, typically being medium containing FBS or platelet lysate. As described elsewhere herein the preferred media for culturing of human MSCs is platelet lysate-supplemented media.

The methods of screening donor cells or donor samples described herein thus permit a user to discriminate between donor cells or samples which are suitable for or conducive to large scale production of MSCs and those which are less suitable. In this manner cells or samples which are unsuitable or less suitable for the intended use can be discarded before extensive efforts are made to use those cells. This method of selecting suitable material allows the user to concentrate efforts on cells and samples which do have capability of being cultured for sufficient population doublings to generate the desired large scale production of MSCs. In order to generate the very large scale production of MSCs envisaged, cells would typically need to be capable of greater than about 10 population doublings before senescence, more preferably greater than about 15 population doublings before senescence, or greater than about 20 population doublings before senescence, or greater than about 25 population doublings before senescence, or greater than about 30 population doublings before senescence, or greater than about 35 population doublings before senescence. Where a cell line arising from a donor sample becomes senescent after less than about 10 population doublings, more so if senescent after less than about 8 or 7 population doublings, it will typically not be suitable for large scale production of MSCs. As described herein the ‘serum switch’ method allows an operator to identify a cell sample as being suitable for or not suitable for very large scale production of MSCs at a relatively early stage, that is without the need for more than about one, two, three or four passages. This ability to discriminate between suitable and unsuitable cell samples is advantageous to the efficiency of very large scale production of MSCs.

### MSCs Grown in Platelet Lysate Secrete High Concentrations of ECM Components

In developing the methods of the invention the inventors identified that surprisingly when MSCs are grown in platelet lysate the tissue culture media becomes viscous as the cells proliferate. Analysis of the viscous media demonstrated that high molecular mass glycoconjugates were present in the media in large amounts (see Examples 4 and 5 herein). The glycoconjugates may include proteoglycans, glycosaminoglycans and/or mucins. The increase in glycoconjugates occurred at the same time as the increase in the viscosity of the media. Further analysis of the media identified that the viscosity of the media was contributed by GAG, proteoglycan, mucin or other high molecular weight molecule or complex of molecules and that the major proteoglycan present may be aggrecan, versican, lumican or biglycan.

The inventors also identified that when MSCs are grown in a culture medium comprising EGF and or FGF a viscous conditioned media was also obtained once the cells became confluent.

The invention thus also provides a method for the preparation of high molecular mass glycoconjugate-enriched conditioned media, the method comprising culture expanding MSCs in a growth medium comprising platelet lysate. Methods for the growth or expansion of MSCs in media comprising platelet lysate are described herein. In an embodiment, the culture medium is DMEM. In an embodiment the platelet lysate is human platelet lysate. In an embodiment the MSCs are MSCs derived from bone marrow, or MSCs derived from dental pulp, or MSCs derived from bone marrow, or MSCs derived from chord tissue, or MSCs derived from chord blood, or MSCs derived from circulating blood, or adipose tissue-derived MSCs. In an embodiment the MSCs are adipose tissue-derived MSCs. In an embodiment the MSCs are human adipose tissue-derived MSCs.

As shown in the Examples herein the viscosity of the media increases as the MSCs proliferate. The inventors have observed that this occurs as the cells proliferate within a given passage, that is as the MSCs become more densely populated within the culture flask.

Accordingly, it is possible for an operator to exert a degree of control over the extent to which the conditioned media is enriched in high molecular mass glycoconjugates, for example by controlling the extent to which the MSCs in the culture are permitted to proliferate. For example, where a less enriched media is desired the growth (proliferation) of the cells will be caused to cease, or the media harvested by the operator, when the cells in the culture have proliferated to about 70% confluence or less. As a further example, where a more enriched or viscous media is desired the growth (proliferation) of the cells will be permitted to continue until greater than about 80% confluence, or greater than about 85% confluence, or greater than about 90% confluence, or greater than about 95% confluence, or about 100% confluence or beyond, so that the media is more concentrated with ECM components.

The invention thus provides a method for the preparation of high molecular mass glycoconjugate-enriched conditioned media and cultured-expanded MSCs, the method comprising culture expanding MSCs in a growth medium comprising platelet lysate. The invention thus provides a combination or composition comprising high molecular mass glycoconjugate-enriched conditioned media and cultured-expanded MSCs.

This combination, which may also be referred to as a composition, may be used for therapeutic purposes in the treatment of a disease, which may herein also be referred to as a disorder or condition, such as an inflammatory disease, disorder or condition. The composition may be used for the manufacture of a medicament for the treatment of such conditions.

It will be apparent that such a combination will be present in the container, such as a tissue culture flask, during the course of the cell growth. A combination of MSCs and high molecular mass glycoconjugate-enriched conditioned media may also be prepared by harvesting the cells and medium from the container after a suitable period of incubation under conditions suitable for the growth of the MSCs.

A combination of MSCs and high molecular mass glycoconjugate-enriched conditioned media may also be prepared by combining in a suitable container isolated MSCs and high molecular mass glycoconjugate-enriched conditioned media substantially free of MSCs. In this manner the combination comprises cells in high molecular mass glycoconjugate-enriched conditioned media different to that which the cells were generated (culture expanded). By preparing a combination of MSCs and high molecular mass glycoconjugate-enriched conditioned media in this manner, that is by using isolated MSCs, the operator is able prepare a combination having a desired number of cells per volume of media. In this manner, for example, a combination having a higher cell density, or number of cells per volume of media, may be generated than would typically be achieved in a combination solely comprised of MSCs in the media in which those same cells were generated. Similarly, a combination having a lower cell density, or number of cells per volume of media, may be generated than would typically be achieved in a combination solely comprised of MSCs in the media in which those same cells were generated. The ability to control the number of cells per volume of media may be advantageous in situations where, for example, a therapeutic advantage may be achieved by administering to a subject a dose having a chosen cell to media ratio. Isolated MSCs may added to a combination of high molecular mass glycoconjugate-enriched conditioned media and MSCs or may be added to high molecular mass glycoconjugate-enriched conditioned media substantially free of MSCs.

The identification herein by the inventors that culture of MSCs in culture media comprising platelet lysate or EGF and/or FGF provides a viscous conditioned media containing high molecular mass glycoconjugate, generally referred to herein as a high molecular mass glycoconjugate-enriched conditioned media, and that this material has beneficial characteristics as described herein, may also be used by an operator to assess the appropriate stage, or assist in assessment of the appropriate stage, at which to harvest cells or conditioned media from culture, such as from a bioreactor. For example, as demonstrated in the Examples herein, conditioned media having a viscosity of about 1.5 cSt or more is enriched in high molecular mass glycoconjugate and provides for improved stability of components secreted from the MSCs, such as VEGF. The Examples also demonstrate that the enriched material is beneficial in the treatment of disease, such as inflammatory conditions and neuropathic pain. As also demonstrated in the Examples, a viscosity of about 1.5 cSt or more is typically achieved in a culture of MSCs grown in the presence of platelet lysate at greater than about 80% confluence. The results herein therefore allow for an operator to use the viscosity of the media in culture, such as in a bioreactor or other culture vessel or container, particularly in culture conditions where the cell density cannot easily or feasibly be determined, as an indication of the appropriate stage at which to harvest the cells and or the conditioned media, depending on the attributes of the media or the cells desired or required by the operator. This may be achieved, for example, by determining the viscosity of a sample of the liquid phase of the culture at a given time and, on the basis of the determined viscosity, harvesting cells or media or continuing the culture, depending on the operator's requirements.

In a typical embodiment the composition comprising high molecular mass glycoconjugate-enriched conditioned media and culture-expanded MSCs are native to each other, meaning that the cells in the composition are cells which have been grown in the high molecular mass glycoconjugate-enriched conditioned media with which they reside in the composition.

In an embodiment the method further comprises separating the MSCs from the culture medium. In the methods of the invention the cells may be separated or harvested from the culture medium by methods known to the skilled addressee. In embodiments the invention thus provides for the preparation of high molecular mass glycoconjugate-enriched conditioned media substantially free of MSCs. In this context a preparation of high molecular mass glycoconjugate-enriched conditioned media would be considered to be “substantially free” of MSCs if it comprises no MSCs or merely remnant contaminant MSCs that may remain after routine steps to harvest or remove adherent cells from cell culture. The media may be considered to be “substantially free” of MSCs if the content of MSCs was at a level expected to have no therapeutic effect if used in a therapy.

The harvested high molecular mass glycoconjugate-enriched conditioned media may be subjected to one or more further processing steps, for example to remove or reduce the presence of contaminant material. In this context, a contaminant material may be any undesirable component of the media, such as cell fragments or debris. Where the high molecular mass glycoconjugate-enriched conditioned media is intended for therapeutic use, a contaminant will also include any component that is incompatible with a pharmaceutical use of the media, for example a component that may be toxic to a recipient animal or which may reduce the therapeutic efficacy of the media or which may reduce the ability of the media to be stored. An example of further processing in this context may include centrifugation or filtering the media.

The harvested high molecular mass glycoconjugate-enriched conditioned media, with or without MSCs, may be stored under any appropriate conditions. Storage is typically frozen at −20° C., −10° C. or −80° C. Alternatively storage may be at 4° C.

The storage may be in separate aliquots or vials such as may be suitable for use in a therapeutic dose in the treatment of a subject. In a further example the media may be stored in an injectable-ready form, such that an operator merely needs to retrieve the material from storage and thaw or warm it to the required temperature before therapeutic use. In an alternative form the storage may be “bulk” storage which may require further dilution or aliquotting for example prior to use, or for example, which may be more suitable for research purposes.

The invention also provides for MSCs cultured on microcarriers, for example in a stirred bioreactor. The MSCs can be cultured on microcarrier beads (90-400 um) which may be uncoated, or coated with particular proteins such as collagen and may be treated to have specific charge distribution on the surface. The microcarriers may be used for the growth of cells in a number of vessels such as spinner flask, microbioreactors, stirred tank, rocking platforms or microgravity. Cells may also be cultured on fibra-cel discs, which may be maintained in a bioreactor as a suspended stirred system or as a static packed bed.

### Compositions and Pharmaceutical Compositions

The methods of the invention include the preparation of compositions, in particular of pharmaceutical compositions. In an embodiment the composition may comprise high molecular mass glycoconjugate-enriched conditioned media substantially free of MSCs. In an embodiment the composition may comprise culture expanded MSCs prepared by a method of the invention. In an embodiment the composition may comprise high molecular mass glycoconjugate-enriched conditioned media and MSCs. In an embodiment the composition may be any of the aforementioned combinations, such as combinations of MSCs and high molecular mass glycoconjugate-enriched conditioned media. A composition of the invention may be used for the preparation of a pharmaceutical composition. The composition may comprise one or more of a pharmaceutically acceptable carrier, diluent, excipient or adjuvant.

A composition of the invention, such as a pharmaceutical composition, may be supplied to the user as a frozen solution. For example, high molecular mass glycoconjugate-enriched conditioned media, culture-expanded MSCs, or a combination thereof, can be stored, at approximately −20° C. until required for use. Alternatively, high molecular mass glycoconjugate-enriched conditioned media, culture-expanded MSCs, or a combination thereof, may be stored at a lower temperature, for example in a freezer at −70° C. to −90° C., or in liquid nitrogen storage, either in the vapour phase or in the liquid phase, until required for use. Compositions comprising cells will typically be stored in liquid nitrogen. In a preferred embodiment the composition comprising high molecular mass glycoconjugate-enriched conditioned media, or the culture-expanded MSCs, or a combination of high molecular mass glycoconjugate-enriched conditioned media and culture-expanded MSCs, is stored in the liquid phase of liquid nitrogen storage. In a preferred embodiment a composition comprising adipose tissue-derived culture-expanded MSCs cells is stored in combination with high molecular mass glycoconjugate-enriched conditioned media. Alternatively, a cell free composition of the invention, such as a pharmaceutical composition, may be supplied to the user as a freeze-dried preparation. For example, high molecular mass glycoconjugate-enriched conditioned media, may be freeze dried and stored, at approximately 4° C., −20° C. or at room temperature until required for use.

In use, such as by the treating physician, clinician, veterinarian, technician, assistant, or farmer, the composition is typically administered to the subject or animal as soon as possible after thawing. The pharmaceutical composition may alternatively be stored, for example on ice or in a refrigerator or in a cool pack, preferably at approximately 2° C. to 5° C. for a short time between thawing and administration. In this context a short time would typically be no more than several hours, such as no more than about half an hour, or no is more than about one hour, or no more than about two hours. As cryoprotectants are typically toxic to the cells and can cause loss of viability if kept thawed, the composition, particularly where it comprises viable cells, is typically injected to the recipient animal as soon as possible after thawing.

A pharmaceutical composition of the invention ray be supplied in a “ready-to-use” form. In such embodiments the user typically requires only thawing to an acceptable temperature for administration before the composition is administered. In such embodiments the composition may be supplied in pre-measured doses, such as a premeasured or pre-determined dose suitable for a given recipient subject or animal, for example pre-determined on the basis of the recipient species, or on the basis of the recipient individual, such as a small breed of dog, compared to a large breed of dog, or a juvenile animal compared to an adult animal. The pre-measured dose may alternatively or additionally be on the basis of the disease or condition being treated or intended to be prevented. A ready-to-use form of the composition may comprise the composition supplied with or in an injectable device, such as a syringe. The injectable device may be capable of delivering a single application to an individual recipient or may be capable of delivering single or multiple applications to multiple recipients. The injectable device may be adjustable, for example to permit delivery of a range of different doses.

In embodiments where the pharmaceutical composition comprises a combination of high molecular mass glycoconjugate-enriched conditioned media, and culture-expanded MSCs, the composition may be supplied to the user as a combination or as separate compositions for combination by the user. It will be understood that reference to a “user” in this context means the individual who actually administers the therapeutic composition to the recipient subject or animal and also means a member of the team or group who is undertaking that administration. For example the user may be any individual who is assisting in the application of the methods of the invention such as a clinician, a doctor, a veterinarian, a farmer, a clinical nurse, a veterinary nurse, a technical assistant, or a farmhand.

As described herein high molecular mass glycoconjugate-enriched conditioned media demonstrates advantageous stability of components, such as VEGF and other molecules secreted from the cultured MSCs, for example when the media is stored at room temperature. As a consequence, the invention also provides for compositions that may optionally be stored without freezing or without refrigeration. Typically a composition stored without freezing or without refrigeration will comprise high molecular mass glycoconjugate-enriched conditioned media but will not comprise MSCs.

A pharmaceutical composition comprising high molecular mass glycoconjugate-enriched conditioned media may thus be provided as a ready to use composition which is suitable for storage at room temperature, which would typically be in the range of about 20° C. to 25° C. In an embodiment the composition may be a composition for topical application, such as to the skin of a subject in need of such application. A composition for topical application may be in any appropriate form, including for example, in the form of a liquid, a gel, a cream or a lotion. When provided in the form of a composition for topical application and which can be stored at room temperature the invention provides for ease of use by a subject, such as ease of self-administration compared to, for example, an injectable composition or a composition that is optimally stored refrigerated or frozen.

A pharmaceutical composition comprising high molecular mass glycoconjugate-enriched conditioned media may be formulated with any appropriate carrier material, such as is suitable for formulation of a therapeutically active ingredient or ingredients for topical application. In the examples herein, the use of a carrier material that does not contain a surfactant allows for higher levels of VEGF to be detected than does the use of a carrier material that does contain a surfactant. Without wishing to be bound by theory the inventors consider this may be due to binding of the VEGF by the surfactant, thereby reducing the detectable VEGF in the composition. The inventors consider that the presence of a surfactant in the pharmaceutical composition, such as by way of inclusion in the carrier material or in the formulating of the composition, may similarly reduce the therapeutically available VEGF in the pharmaceutical composition such that a composition that does not contain surfactant may provide a superior therapeutic product compared to a composition that does contain surfactant. Whilst this explanation is provided with reference to VEGF it is expected that the principle applies to other growth factors and cytokines in the high molecular mass glycoconjugate-enriched conditioned media. In an embodiment the carrier material does not include a surfactant. In an embodiment the pharmaceutical composition does not include a surfactant. In an embodiment the carrier material comprises saline and optionally propylene glycol. In an embodiment the carrier material is Solugel or a formulation similar thereto. In an embodiment the carrier material or pharmaceutical composition comprises one or more thickeners, such as a cosmetic or pharmaceutical thickener(s), for example hydroxyl ethyl cellulose.

### Kits

The invention also provides a kit comprising (a) a pharmaceutical composition selected from the group consisting of (i) high molecular mass glycoconjugate-enriched conditioned media, (ii) a composition comprising culture expanded MSCs, and (iii) a combination of (i) and (ii); and (b) instructions for use of said kit in treatment of an inflammatory disorder, or alleviating pain associated with an inflammatory disorder.

In an embodiment the kit comprises one or more frozen compositions. In an embodiment the kit comprises instructions for combining a composition comprising high molecular mass glycoconjugate-enriched conditioned media and a composition comprising culture expanded MSCs, prior to administration of a combined composition. In an embodiment the kit further comprises one or more injection devices, such as one or more syringes. In an embodiment the injection device contains a composition of the kit.

A composition of the invention may be used in the treatment or prevention of a medical condition conducive to treatment by MSCs or by proteoglycan-enriched conditioned media. For example, as demonstrated herein the composition comprising high molecular mass glycoconjugate-enriched conditioned media was administered to a patient having osteoarthritis with the patient reporting acceptable results. Accordingly, a composition of the invention may be used for the treatment or prevention of an inflammatory disorder or of osteoarthritis in a subject requiring said treatment. A composition of the invention may be used for the alleviation of pain of an inflammatory disorder or of osteoarthritis in a subject requiring said alleviation. As also demonstrated in the Examples a composition of the invention was also beneficial to the patient when administered for treating tennis elbow, a tendon injury, chilblains, tendonitis, golfers wrist, bursitis, muscle or calf tear. The subject may be any animal. In an embodiment the subject is selected from the group consisting of a cat, a dog and a horse. In an embodiment the subject is a human.

### Inflammatory Disorders

The pharmaceutical composition may be administered for the treatment of an inflammatory disorder and/or for alleviating pain associated with an inflammatory disorder in a subject.

Inflammation may arise as a response to an injury or abnormal stimulation caused by a physical, chemical, or biologic agent. An inflammation reaction may include the local reactions and resulting morphologic changes, destruction or removal of injurious material such as an infective organism, and responses that lead to repair and healing. The term “inflammatory” when used in reference to a disorder refers to a pathological process which is caused by, resulting from, or resulting in inflammation that is inappropriate or which does not resolve in the normal manner. Inflammatory disorders may be systemic or localized to particular tissues or organs.

Inflammation is known to occur in many disorders which include, but are not limited to: Systemic Inflammatory Response (SIRS); Alzheimer's Disease (and associated conditions and symptoms including: chronic neuroinflammation, glial activation; increased microglia; neuritic plaque formation; Amyotrophic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis, rheumatoid arthritis, osteoarthritis, prognosis and streptococcus-induced arthritis, spondyloarthropathies, and gouty arthritis), asthma (and associated conditions and symptoms, including: bronchial asthma; chronic obstructive airway disease, chronic obstructive pulmonary disease, juvenile asthma and occupational asthma); cardiovascular diseases (and associated conditions and symptoms, including atherosclerosis, autoimmune myocarditis, chronic cardiac hypoxia, congestive heart failure, coronary artery disease, cardiomyopathy and cardiac cell dysfunction, including: aortic smooth muscle cell activation, cardiac cell apoptosis and immunomodulation of cardiac cell function); diabetes (and associated to conditions, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, is cholesterol gallstones and hepatic fibrosis; HIV infection (and associated conditions, including—degenerative responses, neurodegenerative responses, and HIV associated Hodgkin's Disease); Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis); nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury); neurodegenerative diseases or neuropathological conditions (and associated diseases and conditions, including acute neurodegeneration, induction of IL-I in aging and neurodegenerative disease, IL-I induced plasticity of hypothalamic neurons and chronic stress hyperresponsiveness, myelopathy); ophthalmopathies (and associated diseases and conditions, including diabetic retinopathy, Graves' ophthalmopathy, inflammation associated with corneal injury or infection including corneal ulceration, and uveitis), osteoporosis (and associated diseases and conditions, including alveolar, femoral, radial, vertebral or wrist bone loss or fracture incidence, postmenopausal bone loss, fracture incidence or rate of bone loss); otitis media (adult or paediatric); pancreatitis or pancreatic acinitis; periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis; immunological disorders, including autoimmune diseases, such as alopecia aerata, autoimmune myocarditis, Graves' disease, Graves ophthalmopathy, lichen sclerosis, multiple sclerosis, psoriasis, systemic lupus erythematosus, systemic sclerosis, thyroid diseases (e.g. goitre and struma lymphomatosa (Hashimoto's thyroiditis, lymphadenoid goitre); lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries), septicaemia, acute phase response (e.g. febrile response), general inflammatory response, acute respiratory distress response, acute systemic inflammatory response, wound healing, adhesion, immuno-inflammatory response, neuroendocrine response, fever development and resistance, acute-phase response, stress response, disease susceptibility, repetitive motion stress, tennis elbow, and pain management and response.

In particular embodiments the inflammatory disorder is selected from joint-related inflammatory disorders, corneal inflammation, skin inflammation or wound healing.

In particular embodiments the joint-related inflammatory disorder is arthritis, such as osteoarthritis.

In particular embodiments the compositions of the invention are for the treatment of tennis elbow or for the treatment of a tendon injury, or for the treatment of chilblains or for the treatment of tendonitis, such as of the foot, of for the treatment of golfer's wrist, or for the treatment of bursitis, such as trochanteric bursitis, or for the treatment of Achilles tendonitis, or for the treatment of a muscle tear, such as a calf tear.

### Neuropathic Pain

The pharmaceutical composition may be administered for the treatment of neuropathic pain in a subject. The inventor has identified that compositions of the invention are useful in the treatment of subjects having pain for which there is no discernable clinical cause, such as some forms of neuropathic pain. Neuropathic pain refers to a group of painful disorders characterized by pain due to dysfunction or disease of the nervous system at a peripheral level, a central level, or both. It is a complex entity with many symptoms and signs that fluctuate in number and intensity over time. The three common components of neuropathic pain are steady and neuralgic pain; paroxysmal spontaneous attacks; and hypersensitivity.

Neuropathic pain can be very disabling, severe and intractable, causing distress and suffering for individuals, including dysaesthesia and panaesthesia. Sensory deficits, such as partial or complex loss of sensation, are also commonly seen. In addition, there are significant psychological and social consequences linked to chronic neuropathic pain, which contribute to a reduction in quality of life.

Neuropathic pain is quite common in general medical practice. In some forms, the neuropathic pain is not associated with any discernable clinical causative condition. As an example it is demonstrated herein that the compositions of the invention are effective in alleviating chronic tennis elbow which is considered to be neuropathic pain. In some forms, the neuropathic pain is associated with a discernable clinical condition. The prevalence of trigeminal neuralgia is 2.1 to 4.7 persons per 100,000 of the population, and of painful diabetic neuropathy occurs in 11% to 16% of Type 1 diabetics as well as Type 11 diabetics and post-herpetic neuralgia is found in approximately 34 persons per 100,000 of the population. Treatment of neuropathic pain is not easy. Patients with neuropathic pain do not always respond to standard analgesics such as non-steroidal anti-inflammatory drugs (NSAIDs) and to some extent neuropathic pain is resistant to opiates. The pharmacologic agents best studied and longest used for the treatment of neuropathic pain are antidepressants and anticonvulsants is both of which may have serious side effects.

A composition of the invention may be administered to a subject for treatment of such pain at any appropriate site. Administration may typically be using an appropriate type of injection or it may be by topical application. For example, an injection may be subcutaneous, intramuscular, or directly into an accessible site at or near a site of the pain. As this type of pain may manifest in multiple areas of the subject's body, for example jaw pain and limb or shoulder pain, the administration may be at or near to one site of the pain and remote from another site afflicted by pain. Typically, where multiple sites of the pain occur in a patient, the administration is at or near a site identified as an original or primary site of the pain. As an illustration of this treatment, the examples herein show treatment of golfers wrist and chronic tennis elbow by topical application of a cream or gel comprising high molecular mass glycoconjugate-enriched conditioned media. The topical treatment may involve rubbing the gel or cream on to the affected area, alternatively it may involve applying the cream or gel to a dressing or patch which is then applied to the affected area. A subject being treated may be administered a single application of a composition of the invention, or may preferably be administered multiple applications.

The invention will now be described in more detail, by way of illustration only, with respect to the following examples. The examples are intended to serve to illustrate this invention and should not be construed as limiting the generality of the disclosure of the description throughout this specification.

### EXAMPLES

### Example 1. Screening of Canine Cells from Different Donors

### Processing of Adipose Tissue

Samples (10 g) of falciform adipose tissue was collected from five female dogs during routine desex procedures. The five samples of adipose tissue were processed separately. Adipose tissue was rinsed with saline and then minced finely using scissors and mixed with 20 mls of Dulbecco's Modified Eagle's Medium (DMEM, Sigma). Collagenase (Sigma) was added to a final concentration of 0.05% wt/vol and the sample was incubated at 37° C. for 90 minutes. During the incubation the samples was gently inverted by hand every 1.5 minutes.

Following collagenase treatment the samples were aseptically filtered through stainless steel mesh (700 μm pore size), transferred to 50 ml centrifuge tubes and centrifuged at 500 g for 15 minutes.

The floating cells and the supernatant were discarded and the pelleted cells were gently mixed with a pasteur pipette and transferred to 15 ml centrifuge tubes.

The cells were then washed in DMEM to remove collagenase. DMEM was added to each tube to a final volume of 14 mls and centrifuged at 500 g for 10 minutes. The supernatant was discarded and the pelleted cells were gently resuspended in 4 mls of DMEM and mixed with a pasteur pipette.

### Expansion of Cells in Fetal Calf Serum

Aliquots (0.5 mls) of the cell suspensions from each donor were transferred to tissue culture flasks containing DMEM plus 10% fetal calf serum and incubated in a CO₂ incubator at 37° C. until a confluent cell monolayer was present (7 to 10 days). Cells were stripped with 3 mls of TrypLE Express (Invitrogen), decanted into 50 ml centrifuge tubes and centrifuged at 500×g for 10 minutes. Cells were placed into new tissue culture flasks containing DMEM plus 10% fetal calf serum.

### Transfer of Cells in to Allogeneic Serum

The cells cultured in fetal calf were stripped when confluent and placed in to new flasks containing DMEM plus 10% allogeneic canine serum and incubated for 7 to 14 days. Flasks were examined using a microscope and the confluency of the cells scored on a range of 0% to 100%.

### Cell Proliferation in Canine Serum

As described in the following paragraphs, cells from two of the six donors proliferated rapidly when transferred to the canine serum. The cells from the other 4 donors did not grow in the canine serum.

### Manufacturing of Product

Cells from each of the 6 donors were expanded in media containing 10% FBS up till, passage 2, at which point the cells in all the 6 lines had undergone 7.5-8.5 population doublings. At passage 3 the cells were seeded out in media containing 10% allogeneic canine serum and grown to confluence or up to 14 days without any media changes. It was observed that two of the Donors (1 and 2) cells grew to confluence whereas the remaining 4 donors cells did not achieve confluence in 14 days. Upon harvesting the cells it was observed that the cells from Donors 1 and 2 had undergone an additional 2 population doublings. However, the cells from the other donors had undergone less than 1 population doubling whilst in the media containing 10% allogeneic canine serum.

Cells from all 6 donors were expanded from passage 2 in 10% FBS culture media (without changing over to canine serum). Cells were grown to confluence and passaged multiple times while the culture was observed to still have typical MSC morphology and observed to be proliferating and achieving confluence. It was observed that cells from Donor 1 and 2 proliferated in typical manner to passage 6 and had a cumulative population doubling of 13-14. Therefore, from these donors (1 and 2) cell therapy products could be manufactured from Passage 5 or 6 cells, which have undergone more than 13 doublings. However, cells from other donors stopped proliferating at passage 5. The morphology of cells derived from those four donors whose cells stopped proliferating, being the same cell populations (donors) whose cells had performed relatively poorly in the media containing 10% allogeneic canine serum, had also visibly deteriorated at passage 5. Therefore, any cell therapy product manufactured from these donors would only be at passage 4, and would have cumulative population doubling of 9-10.

Therefore passaging cells in FBS and then testing them by exposing them to an allogeneic serum could be used as a method for predicting the ability to expand cells to higher passage numbers which would reduce cost of goods per vial and also reduce research and development time. As a short-hand terminology this method of screening and variations thereof may herein be referred to as a ‘serum switch’ method.

### Example 2. Production of Platelet Lysate

Blood was collected in to blood collection bags with citrate used as the anticoagulant, according to methods known in the art. The blood was dispensed into centrifuge tube and centrifuged at 200 g for 20 min. The top layer containing the platelets was collected and subjected to 4 cycles of freeze thawing from liquid nitrogen in to a 37° C. waterbath. The lysed platelets were then serum converted by the addition of thrombin and calcium chloride, then centrifuged for 10 min at 4000 g and the pelleted cell fragments discarded.

The platelet lysate was then filter sterilised (0.22 micron) and stored at −80° C. until required.

### Example 3. Production of Human Adipose Derived Stem Cells

### Processing of Adipose Tissue

Liposuction was used to collect approximately 200 grams of adipose tissue from the abdomen and or thighs of each patient. The lipoaspirate was processed immediately after collection by washing with warmed (37° C.) sterile Ringers Solution (Baxter) and then digesting by adding sterile collagenase to a final concentration of 0.05% wt/vol. The sample was incubated at 37° C. for 20 minutes with gentle mixing at 100 rpm on an orbital mixer, filtered through a 800 micron mesh, transferred to centrifuge tubes, and centrifuged at 500 g for 15 minutes.

The floating cells and the supernatant were discarded and the pelleted cells were gently mixed with a pasteur pipette and transferred to a 15 ml centrifuge tube.

The cells were then washed in DMEM to remove collagenase. DMEM was added to a final volume of 14 mls and the sample centrifuged at 500 g for 10 minutes. The supernatant was discarded and the pelleted SVF cells were gently resuspended in 4 mls of DMEM and mixed with a pasteur pipette.

### Expansion of Cells

Aliquots (0.5 mls) of the cell suspension were transferred to tissue culture flasks containing DMEM plus 5% human platelet lysate and incubated in a CO₂ incubator at 37° C. until a confluent cell monolayer was present (7 to 10 days). Cells were stripped with 3 mls of TrypLE Express (Invitrogen), decanted into 50 ml centrifuge tubes and centrifuged at 500×g for 10 minutes. Cells were passaged further until they had doubled approximately 8 times. The passaged cells were then stripped and centrifuged.

### Cryopreservation of Cells

The pelleted cell samples were resuspended in CryoStor CS10 (Biolife Solutions, USA) and transferred to cryovials in 2 ml aliquots and the cryovials were frozen in a controlled rate freezing device at approximately 1° C. per minute in a −80° C. freezer for 24 hours and then transferred to a liquid nitrogen dewar for long term storage.

### Example 4. MSCs Grown in Platelet Lysate Secrete High Concentrations of ECM Components

### Production of Conditioned Media

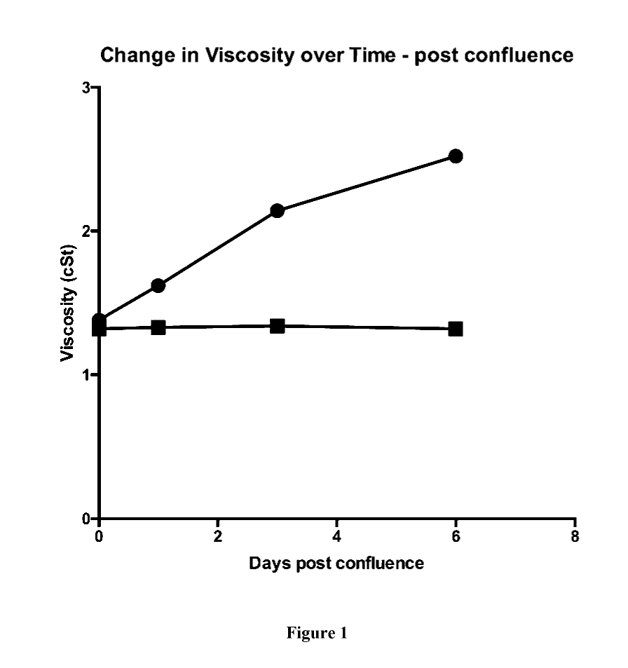

The media from cells grown in Example 3 after 3 passages was collected and analysed for viscosity. Cells were also grown in 10% fetal calf serum as a control. The viscosity of the culture media increased as the cells became more confluent (FIG. 1), marginally in the cultures grown in FCS and substantially in the cultures grown in platelet lysate.

Analysis of Viscous Conditioned Media

Samples of the conditioned media were sent to the Australian Proteome Analysis Facility (APAF) for analysis. The analysis is detailed in Example 5.

### Example 5. To Investigate the Gradual Increase in Viscosity of the Cell Culture Medium: Evidence for Tissue Culture Supernatant from Mesenchymal Stem Cells Grown in Platelet Lysate Containing Extracellular Matrix Component(s) and or Mucins

The research was based on the observation that growth of adipose derived stem cells in 5 or 10% Platelet Lysate culture medium increases the viscosity of the culture medium over time. Two outcomes were approached: quantification of the culture medium viscosity and identification of the cause of the increase in viscosity, as described in the following paragraphs.

### Viscosity Measurement

Viscosity was measured using a Cannon-Fenske Routine capillary viscometer (Cannon Instrument Company, serial number T209), which measures kinematic viscosity, as per the manufacturer instructions. Briefly, the viscometer was filled with approximately 5 ml of sample (culture medium or re-suspended precipitate) by suction and allowed to equilibrate to 40° C. for at least 10 mins in a water bath. Once at temperature, the sample was pulled up to the first marked point by suction and the time taken for the meniscus to move between the first and second marked points was measured (the efflux time). The efflux time was then multiplied by the calibration constant (0.009001 cSt/s) to obtain the kinematic viscosity in centistokes (cSt). Viscosity was plotted over six days of cell growth. The viscosity increased relative to growth in Fetal Calf Serum medium at each determination (FIG. 1).

The viscosity component in the culture medium was investigated as described in the following paragraphs.

Precipitation with Acetone and Trichloroacetic Acid

In a 50 ml falcon tube, 5 mL (1 volume) of culture medium was mixed with 20 mL (4 volumes) of acetone chilled to −30° C. The mixture was agitated by vortex then allowed to precipitate overnight at −30° C. The precipitate (PT1) was collected by centrifugation at 4600 g, 4° C. for 20 minutes. The supernatant (SN1) was removed and the tube left upside down to drain the precipitate for ˜2 mins, taking care to avoid complete drying of the pellet. PT1 was re-suspended in 100 mM ammonium bicarbonate pH 7.8 for viscosity measurements and enzymatic digestions.

Acetone precipitation (80%, −30° C.) precipitated the viscosity component from the culture medium indicating that it is of a high molecular weight, which was estimated to have the similar viscosity of hyaluronic acid at a concentration of about 0.2-0.3 mg/ml.

Monosaccharide Analysis by High Performance Anion Exchange Chromatography with Pulsed Amperometric Detection (HPAEC-PAD)

A stock solution of 25% (v/v) trifluoroacetic acid (TFA) was prepared. To 50 μL of sample, 150 μL, of MilliQ water and 300 μL of 25% (v/v) TFA were added. Samples were incubated at 100° C. for 4 hours with shaking. Following incubation, samples were dried by vacuum centrifugation and reconstituted in 200 μL of 100 μM 2-deoxy-D-glucose (internal standard).

Samples were analysed on a Dionex HPLC instrument, on a CarboPac PA1 carbohydrate column, with pulsed amperometric detection. The neutral monosaccharides were eluted isocratically with 16 mM NaOH. The acidic monosaccharides were eluted using gradient elution of sodium acetate in 100 mM NaOH. The identity of peaks was determined by elution time by comparison against known standards of neutral (fucose, galactose, glucose, mannose), amino (N-acetylgalactosamine, N-acetylglucosamine), and acidic (N-acetylneuraminic acid, glucuronic acid) monosaccharides.

Monosaccharide analysis of the acetone precipitated viscosity component indicated the presence of the monosaccharides glucuronic acid, N-acetylglucosamine and some galactose, which are components of the glycosaminoglycans (GAGs) that are linked to proteoglycans as major components of the extracellular matrix (ECM). From the compositional analysis this would indicate either hyaluronic acid, keratan sulfate and/or chondroitin sulphate GAGs and/or mucins and/or other glycoconjugates in the material.

The viscosity of acetone precipitate material was analysed following enzymatic treatment with Keratanase (Kase), Hyaluronidase (Hase), Chondroitinase ABS (Csase), Endo-β-galactosidase (EBG), each separately at 37° C. for 3 days. Control medium was incubated in 100 mM Ammonium bicarbonate, 37° C., 3 days no enzyme. The results of kinematic viscosity showed a reduction in viscosity with the enzymes Keratanase, Hyaluronidase and Chondroitinase, indicating that the enzymes did act upon their respective GAG's within the precipitate. This result supported the deduction that the viscosity component is a GAG, proteoglycan, mucin or other high molecular weight molecule or complex of molecules.

Enzymatic digestion of standard solutions of Chondroitin sulphate, Aggregan and Hyaluronic acid with their respective enzymes of Chondroitinase, Keratanase and Hyaluronidase was carried out as set out in Table 1.

TABLE 1
Digestion conditions optimized for digestion of Chondroitin sulphate, keratan
sulphate (component of Aggrecan) and Hyaluronic acid.

| | Enzyme | | |
|---|---|---|---|
| Enzyme | concentration | Reaction buffer | Standard |
| Chondroitinase ABC | 5 μl | 100 mM | Chondroitin |
| (20 mU/μl) | | Ammonium acetate | sulphate (5 μg) |
| | | pH 8 | Aggrecan (10 μg) |
| Keratanase | 2 μl | 100 mM Tris-HCl | Aggrecan (10 μg) |
| (50 mU/μl) | | pH 7 | |
| Hyaluronidase | 5 μl | 100 mM Sodium | Select-HA 50K (20 μg) |
| (2 U/μl) | | acetate pH 5 | |

Reaction volume = 20 μl, 37° C. incubation overnight

The GAG disaccharide digestion products were detected using either LC-MS or direct MS and their composition confirmed by MS² fragmentation from components separated using a porous graphitized carbon column eluting with ammonium bicarbonate in acetonitrile and negative ion mass spectrometry with a Thermo Velos ion trap mass spectrometer.

The cell secretion samples were produced from cells grown in alpha MEM media plus 10% platelet lysate (serum converted) (Stemulate, Cook Regentec, Bloomington, Ind., USA) and 2. DMEM media plus 5% platelet lysate (PLTMax, Millcreek, Rochester, Minn., USA) plus 2 IU/mL heparin.

To identify the secreted proteoglycans present, concentrated cell secretion/media samples were immobilised onto PVDF membrane under vacuum, treated with GAG degrading enzymes, Keratanase (Kase), Hyaluronidase (Hase), Chondroitinase ABS (Csase), Endo-β-galactosidase (EBG) and analysed by MS.

The second stage MS spectra of the digestion products confirmed that the mass (m/z 460²⁻ and 380²⁻ m/z) matched the sulphated chondroitin sulphate fragmentation spectra of the aggrecan standard, thus confirming the presence of chondroitin sulphate in the conditioned media.

Chondroitin sulphate is a GAG component of Aggrecan and also of other proteoglycans such as Versican. The presence of Chondroitin sulphate confirms the presence of one or a mixture of such proteoglycans.

The glycan mass spectrometry data was determined on the sugars digested from a precipitate taken from an acetone precipitation and the levels of Chondroitin sulphate were lower than expected. However when samples (50 μl) were dot blotted under vacuum onto PVDF membrane using a Bio-Rad dot blotting apparatus and stained with Alcian blue (0.1% Alcian blue in 0.1% acetic acid) before and after acetone precipitation and staining intensity compared, the staining intensity (darker blue staining colouration) was higher in the cell secretion samples before acetone precipitation (results not shown) suggesting not all charged macromolecules (i.e. proteoglycans) were precipitated by acetone.

Proteomic analysis was performed on the total cell secretions and on the high molecular weight proteins separated by Sepharose size fractionation. Trypsin digested peptides were analyzed using on-line capillary reversed phase (RP) (ProteCol C18, 300 μm ID, 10 cm, 5 μm particle size, 300 Å pore size) liquid chromatography (LC) mass spectrometry (MS) in positive polarity mode with either resonance activation-collision induced dissociation (CID). The column was equilibrated in 100% solvent A (0.1% (v/v) formic acid and the peptides separated by a two-step gradient of 0%-30% solvent B (0.1% (v/v) formic acid in acetonitrile), followed by 30%-60% solvent B. The peptide mixtures were reconstituted in 15 μl of deionized water and 5 μl was injected onto the column.

The extracellular matrix proteoglycans that were detected were Lumican, Versican and Biglycan. Chondroitin sulphate is a GAG chain component of Versican as well as Aggrecan. Aggrecan was not detected in the proteomic analysis, however the proteomic analysis was sub-optimal for proteoglycans and the extensive sugar chains on Aggrecan could have inhibited the detection of the protein components.

An ELISA kit with Aggrecan specific antibodies confirmed varying levels of 50-900 pg/mL of Aggrecan in conditioned media from both media types (with and without heparin) and across passages 1-10 (results not shown). Seven different adipose tissue donors were tested and aggrecan was detected from conditioned media from all 7 donors.

Chondroitin sulphate was confirmed to be present in the conditioned media. Chondroitin sulphate is a component of both proteoglycans Versican and Aggrecan. Proteomic analysis detected Versican, lumican and biglycan, but did not confirm the presence of aggrecan, potentially the proteomic analysis was inhibited by the extensive sugar chains on Aggrecan. Aggrecan was confirmed to be present by an Aggrecan antibody ELISA assay in conditioned media of both media types and was not present in matching blank growth media.

### Example 6. Industrial Scale Production of Human Allogeneic Adipose Derived MSCs

### Donor Selection

Adipose tissue was collected from human donors as described in Example 3. Cells from each donor were screened according to Example 1. Cells that showed the appropriate properties, such as acceptable performance in the ‘serum switch’ screening method, were selected for large scale expansion.

### Expansion of Cells

Cells were expanded and passaged as described in Example 3. Ten layer tissue culture factories were used for passaging the cells. Cells continued to be passaged until passage 8 without the cells slowing down or appearing to become senescent.

Calculated Number of Vials that can be Produced from the One Donor

Table 2 shows that over 332 million vials each containing 5 million cells can be produced from the one donor using the methods of the invention.

TABLE 2
Calculated number of vials that can be produced from the one donor.

| | | No. of | | No. of |
|---|---|---|---|---|
| | Cells seeded | doublings | Cells output | vials product |
| Passage 0 | 173.5 | 0.13 | 189.86 | 38 |
| Passage 1 | 189.9 | 3.47 | 2103.81 | 421 |
| Passage 2 | 2103.8 | 3.59 | 25334.08 | 5,067 |
| Passage 3 | 25334.1 | 3.2 | 232809.73 | 46,562 |
| Passage 4 | 232809.7 | 2.5 | 1316970.73 | 263,394 |
| Passage 5 | 1316970.7 | 3 | 10535765.85 | 2,107,153 |
| Passage 6 | 10535765.9 | 2 | 42143063.41 | 8,428,613 |
| Passage 7 | 42143063.4 | 2.5 | 238397167.31 | 47,679,433 |
| Passage 8 | 238397167.3 | 2.8 | 1660294306.33 | 332,058,861 |

### Example 7. An Alternative Tissue Culture Media for the Expansion of MSCs

Human cells were isolated from adipose tissue and cultured as described in Example 3. After two passages the media was replaced with High Glucose DMEM, 10% fetal calf, 20 ng/ml epidermal growth factor (EGF), 20 ng/ml basic fibroblast growth factor and 2% B27 Supplement (Life Technologies). Once the cells became confluent the conditioned media was viscous.

### Example 8. Treatment of a Human Patient with Osteoarthritis with Allogeneic Cells and High Molecular Mass Glycoconjugate-Enriched Conditioned Media

### Processing of Adipose Tissue

Liposuction was used to collect 832 grams of adipose tissue from the abdomen and or thighs of a patient. The lipoaspirate was processed immediately after collection by washing with warmed (37° C.) sterile Ringers Solution (Baxter) and then digesting by adding sterile collagenase to a final concentration of 0.05% wt/vol. The sample was incubated at 37° C. for 20 minutes with gentle mixing at 100 rpm on an orbital mixer, filtered through a 800 micron mesh, transferred to centrifuge tubes, and centrifuged at 500 g for 15 minutes. The floating cells and the supernatant were discarded and the pelleted cells were gently mixed with a pasteur pipette and transferred to a 15 ml centrifuge tube.

The cells were then washed in DMEM to remove collagenase. DMEM was added to a final volume of 14 mls and the sample centrifuged at 500 g for 10 minutes. The supernatant was discarded and the pelleted SVF cells were was pooled and divided into 8 separate freezing bags. The cells were cryopreserved in Cryostor 10 in volumes of 11.5-12.5 ml per bag.

### Expansion of Cells

A bag of cells was thawed and seeded in a T175 tissue culture flask containing Alpha MEM and 10% human platelet lysate. The platelet lysate had been treated by the addition of thrombin and calcium chloride, prior to centrifugation to remove fibrinogen. Cells were incubated in a CO₂ incubator at 37° C. until a confluent cell monolayer was present (7 to 10 days). Cells were passaged 4 times as described in Example 3. The passaged cells were then stripped and centrifuged. The conditioned media from the final passage was collected and used for cryospreservation of the cells.

### Cryopreservation of Cells

The pelleted cell samples were resuspended in 1.8 mL of conditioned media and 0.2 mL of bloodstor-100 cryopreservative fluid (Biolife Solutions, USA) and transferred to cryovials in 2 ml aliquots and the cryovials were frozen in a controlled rate freezing device at approximately 1° C. per minute in a −80° C. freezer for 24 hours and then transferred to a liquid nitrogen dewar for long term storage.

### Treatment of the Patient

A 52 year old patent with grade 3 osteoarthritis of the knee was treated with a single intra-articular injection of expanded allogeneic cells and high molecular mass glycoconjugate-enriched conditioned media. The vial containing the cells and conditioned media was thawed at room temperature and immediately injected in to the knee joint. The injection consisted of 3.9 million cells in 1.8 mL of high molecular mass glycoconjugate-enriched conditioned media and 0.2 mL of bloodstor-100 cryopreservative fluid.

The patient was examined 1 week after the injection and reported a significant reduction in pain. The patient was so pleased with the outcome that they asked if they could have an injection in the other knee.

### Example 9. Stability of Secretions

### Preparation of Secretions

Human adipose derived MSCs (obtained as described in Example 8) were cultured in 10 layer cell factory flasks in either alphaMEM plus 10% platelet lysate (serum converted) (Stemulate, Cook Regentec, Bloomington, Ind., USA) or in alphaMEM plus 10% fetal calf serum. The media was not changed and the cells were kept in culture for 4-7 days until the cells were 80 to 100% confluent. The supernatant was harvested and was observed to be viscous from the platelet lysate media but was not viscous from the fetal calf serum media. The supernatant was not filtered as filtering was observed to reduce the viscosity.

### Analysis of VEGF by ELISA

Vascular endothelial growth factor (VEGF) was measured using an ELISA (R&D Systems, Minneapolis, Minn., USA).

Analysis of secretions by Bioplex

Fourteen different cytokines and growth factors were measured using a Bioplex system (Biorad).

### Stability Testing

Samples of the secretions were stored in test tubes at 22° C. and tested at regular intervals. Samples were also stored at −80° C. as a control. Samples stored at 22° C. were compared to the −80° C. controls.

### Results

The levels of VEGF measured by ELISA in the secretions over a 6 month period did not decline significantly (FIG. 2).

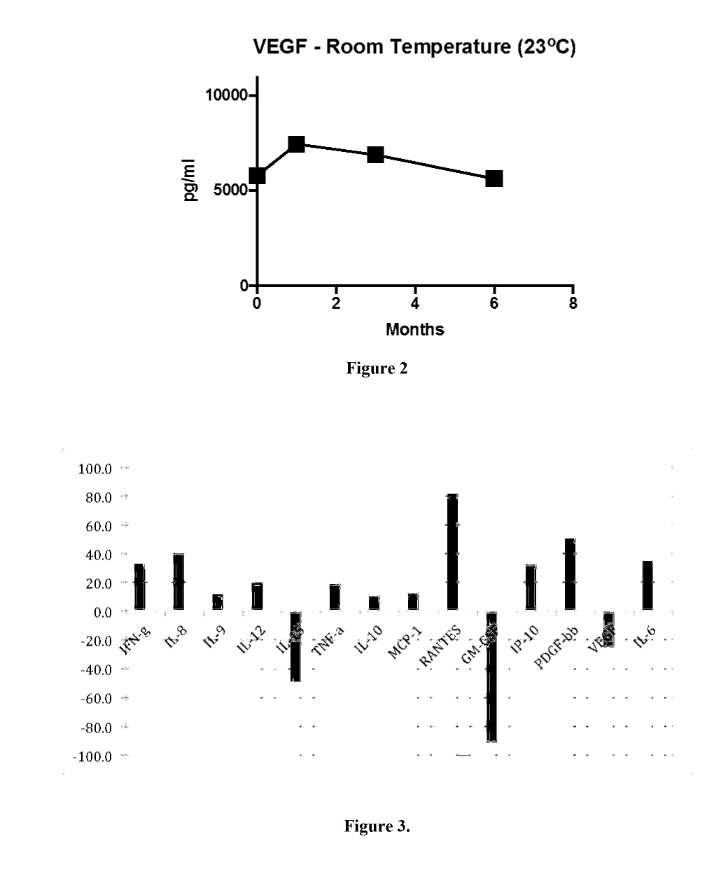

Fourteen different cytokines and growth factors were measured in viscous conditioned media and non-viscous conditioned media, which was stored at room temperature for a period of 5 months. The percentage change in the level of each cytokine or growth factor, compared to the level at the starting time point was calculated and it demonstrates that the different cytokines and growth factors measured were more stable in the viscous conditioned media than the non-viscous conditioned media (FIG. 3).

### Significance

It is surprising that the levels of VEGF remained stable at room temperature for more than a few days. The literature teaches that cytokines such as VEGF and the majority of others analysed in this example are not stable unless stored frozen at low temperatures (Kisand et al., Porter et al.).

### Example 10. Measurement of Viscosity

Adipose derived cells were prepared as described in Example 8. Cells were cultured in either alphaMEM plus 10% platelet lysate (serum converted) (Stemulate, Cook Regentec, Bloomington, Ind., USA) or in DMEM plus 10% fetal calf serum. Cells were cultured through to passage 4 or 5 and the conditioned media was harvested when the cells were between 50% and 100% confluent. The viscosity of the conditioned media was measured using a cannon-feske U viscometer.

### Results

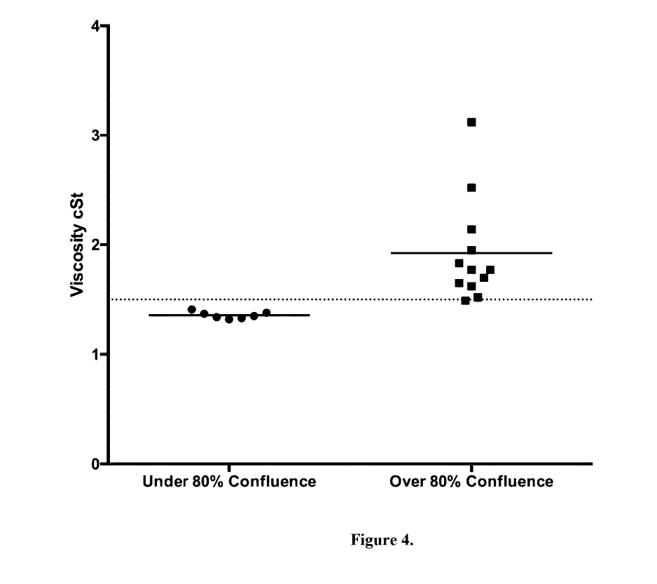

All cultures that were allowed to reach approximately >80% confluence had a viscosity greater than 1.5 cSt. Whereas all cultures that were harvested before 80% confluence had a viscosity below 1.5 cSt (FIG. 4).

### Example 11. Treatment of Tennis Elbow with a Composition Comprising Secretions from Passaged Human Adipose Derived Adherent Cells

Secretions were produced from passaged human adipose derived adherent cells as described in Example 9, with growth of the cells in alpha MEM plus 10% platelet lysate.

Secretions were mixed 1 in 10 with Solugel (Johnson &Johnson) to produce a gel for topical application. A 72 year old woman with tennis elbow that had been ongoing for 2 years was treated by applying the gel to the elbow twice a day. After 5 days the woman reported a significant reduction in pain and was able to play bowls where prior to the treatment the pain in her elbow had precluded her from such activities.

A second female patient, aged 77 years, with Tennis Elbow of the medial side and outer side was treated by application of the gel twice a day. The patient reported a better feel in 4 to 5 days. After 2.5 weeks the patient ceased using the gel because the affected elbow “feels good”. The patient reported that there was no lapse in how the elbow felt 3 weeks after ceasing to apply the gel. The patient reports that she is completely cured. The improvement has allowed the patient to increase their physical capacity, as evidenced by, prior to commencing the secretions treatment, being restricted by pain to bowling 10 ends to being able, after the secretions treatment, to do 21 ends.

### Example 12. Treatment of Tendon Injuries with a Composition Comprising Secretions from Passaged Human Adipose Derived Adherent Cells

The following subjects with a range of conditions were treated using secretions from passaged human adipose derived cells mixed with Solugel prepared as described in Example 10, with growth of the cells in alphaMEM plus 10% platelet lysate. In all cases application of the gel was by topical application to the skin of the affected area, typically twice a day.

### Chilblains and Tendonitis of the Foot

Seventy-five year old female having chilblains and tendonitis of the foot. Pain and inflammation disappeared after 4 days of treatment.

### Golfers Wrist

Seventy-five year old female treated for golfers wrist. Pain and inflammation disappeared after 4 days of treatment.

### Trochanteric Bursitis.

Trochanteric Bursitis. Pain and inflammation disappeared after 4 days of treatment.

### Achilles Tendonitis.

A 74 year old patient with Achilles Tendonitis, which had been an ongoing issue for number of years was treated with the gel containing secretions. The patient reported that the discomfort has disappeared after using the gel for 3 days. The patient has continued to apply the gel twice a day for 3 weeks and the pain has not returned.

### Calf Tear

A 52 year old patient with a recent calf tear was treated with the secretions gel. The patient reported that upon application the secretions gel gave a soothing benefit similar to menthol and that the next morning stiffness in leg, which had affected the patient prior to commencing the treatment was not noted. The patient reapplied the secretions gel each night and reported that the injury is healing well. The patient reported that, during the treatment, one missed nightly application of the secretions gel was followed the next morning by increased stiffness of the affected area of the leg.

### REFERENCES

Cesaretti, M., E. Luppi, F. Maccari, and N. Volpi. 2003. A 96-well assay for uronic acid carbazole reaction. Carbohydrate Polymers. 54:59-61.
Kilcoyne, M., J. Q. Gerlach, M. P. Farrell, V. P. Bhavanandan, and L. Joshi. 2011. Periodic acid-Schiff's reagent assay for carbohydrates in a microtiter plate format. Analytical Biochemistry. 416:18-26.
Kisand, K., Kerna, I., Kumm, J., et al. (2010). Impact of cryopreservation on serum concentration of matrix metalloproteinases (MMP)-7, TIMP-1, vascular growth factors (VEGF) and VEGF-R2 in Biobank samples. Clinical Chemistry and Laboratory Medicine, 49(2), pp. 229-235.
Porter A E, Auth J, Prince M, Ghidini A, Brenneman D E, Spong C Y. Optimization of cytokine stability in stored amniotic fluid. American Journal of Obstetrics and Gynecology [2001, 185(2):459-462]

1. A method for treating an inflammatory condition in a subject, the method comprising administering to said subject a therapeutically effective amount of a high molecular mass glycoconjugate-enriched conditioned media.

2. The method according to claim 1, wherein the inflammatory condition is osteoarthritis.

3. The method according to claim 1, wherein the method further comprises administering a therapeutically effective amount of culture-expanded MSCs.

4. The method according to claim 1, wherein the method comprises administering a composition comprising culture-expanded MSCs and high molecular mass glycoconjugate-enriched conditioned media.

5. The method according to claim 1, wherein the high molecular mass glycoconjugate-enriched conditioned media is prepared by culturing MSCs in media comprising platelet lysate.

6. Use of platelet lysate for the preparation of high molecular mass glycoconjugate-enriched conditioned media.

7. The use according to claim 6, wherein the platelet lysate is human platelet lysate.

8. A method for the preparation of high molecular mass glycoconjugate-enriched conditioned media, the method comprising culturing mesenchymal stem cells in media comprising platelet lysate.

9. The method according to claim 8, wherein the MSCs are adipose tissue-derived MSCs.

10. The method according to claim 8, wherein the platelet lysate is human platelet lysate.

11. The method according to claim 8, wherein the media comprises about 5% to about 10% v/v platelet lysate.

12. The method according to claim 8, wherein the enriched media comprises one or more of a proteoglycan, a glycosaminoglycan and a mucin.

13. The method according to claim 8, wherein the enriched media comprises one or more of keratan sulphate, dermatan sulphate, heparin sulphate, lumican, versican, biglycan, chondroitin sulphate or aggrecan.

14. The method according to claim 8, further comprising the step of removing the cells from the media.

15. A composition comprising high molecular mass glycoconjugate-enriched conditioned media.

16. The composition according to claim 15, wherein said composition is prepared by culturing MSCs in media comprising platelet lysate.

17. A composition comprising culture-expanded MSCs and high molecular mass glycoconjugate-enriched conditioned media.

18. The composition according to claim 15 or 17, wherein said composition is a pharmaceutical composition.

19. The composition according to claim 17, wherein the cells are not adhered in a container containing said composition.

20. The composition according to claim 15 or 17, when prepared according to the method of claim 8.

21. A pharmaceutical composition comprising high molecular mass glycoconjugate-enriched conditioned media according to claim 15 or 17 and a pharmaceutically acceptable carrier, excipient or adjuvant.

22. A method of screening a sample of MSCs for suitability for use in large scale production of cultured MSCs, the method comprising the steps of (i) culturing cells of said sample in a culture medium comprising platelet lysate or FCS for 1, 2, or 3 passages and (ii) culturing said cells or an aliquot thereof after step (i) in a culture medium comprising allogeneic serum, wherein continued proliferation of cells in said culture medium comprising allogeneic serum is indicative of a sample suitable for use in the production of large scale numbers of cultured MSCs.

23. The method according to claim 22, wherein suitability for use in large scale production of cultured MSCs comprises the capacity for at least 15 population doublings before senescence.

24. The method according to claim 22, wherein suitability for use in large scale production of cultured MSCs comprises the capacity for at least 20 population doublings before senescence.

25. The method according to claim 22, wherein suitability for use in large scale production of cultured MSCs comprises the capacity for at least 25 population doublings before senescence.

26. The method according to claim 22, wherein suitability for use in large scale production of cultured MSCs comprises the capacity for at least 35 population doublings before senescence.

27. The method according to claim 22, wherein the sample of MSCs is an adipose tissue-derived sample of MSCs.

28. The method according to claim 22, wherein the sample of MSCs is selected from human, canine, equine and feline MSCs.

29. A method of screening a sample of MSCs for unsuitability for use in large scale production of cultured MSCs, the method comprising the steps of (i) culturing cells of said sample in a culture medium comprising platelet lysate or FCS for 1, 2, or 3 passages and (ii) culturing said cells or a portion thereof from step (i) in a culture medium comprising allogeneic serum, wherein failure of the cells to proliferate in said culture medium comprising allogeneic serum is indicative of a sample unsuitable for use in the production of large scale numbers of cultured MSCs.

30. The method according to claim 29, wherein failure of the cells to reach confluence is indicative of failure of the cells to proliferate.

31. A method for large scale production of cultured MSCs, the method comprising the steps of (i) obtaining a cell sample or tissue sample comprising MSCs, (ii) culturing at least a portion of said sample in a culture medium comprising platelet lysate or FCS for 1, 2, or 3 passages to provide a cultured cell population, (iii) culturing a portion of said cultured cell population from step (ii) in a culture medium comprising allogeneic serum, wherein upon continued proliferation of cells in said culture medium comprising allogeneic serum (iv) culturing at least a portion of said cultured cell population from step (i) or step (ii) in a culture medium comprising platelet lysate or FCS for additional passages to provide a large scale preparation of cultured MSCs.

32. The method according to claim 31, wherein the cell sample is a cell suspension comprising stromal vascular fraction of adipose tissue.

33. The method according to claim 31, wherein the cell sample comprises isolated MSCs.

34. The method according to claim 31, wherein the cell sample comprises culture expanded MSCs.

35. The method according to claim 31, wherein the cells of at least one of steps (i) to (iv) are cells that have been frozen.

36. The method according to claim 31, wherein step (iv) comprises culturing said cell population for 10 or more additional population doublings; or for 15 or more additional population doublings; or for 20 or more additional population doublings; or for 25 or more additional population doublings; or for 35 or more additional population doublings.

37. The method according to claim 31, wherein the method further comprises harvesting culture expanded MSCs after said additional passages and, optionally, aliquoting said harvested cells into individual containers constituting a therapeutic dose of MSCs.

38. The method according to claim 37, wherein a therapeutic dose of MSCs comprises between about 2 million and about 200 million MSCs.

39. The method according to claim 37, wherein a therapeutic dose of MSCs comprises about 5 million MSCs.

40. A pharmaceutical composition comprising MSCs prepared according to the method of claim 31.

41. A method for the preparation of a purified population of culture-expanded adipose tissue-derived mesenchymal stem cells, the method comprising steps of:
(i) obtaining a sample of adipose tissue from a subject,
(ii) incubating said adipose tissue under suitable conditions to at least partially digest said adipose tissue, wherein said conditions comprise incubation in a buffer comprising calcium at a concentration of between 50 mg/L to 500 mg/L and in the presence of collagenase at a concentration of between 0.2% wt/vol to 0.02% wt/vol,
(iii) centrifuging said incubated adipose tissue to obtain stromal vascular fraction (SVF),
(iv) seeding said SVF cells into tissue culture at a desired seeding density,
(v) incubating said culture under appropriate conditions in serum-supplemented media to at least 85% flask confluence,
(vi) harvesting MSCs from said culture,
(vii) passaging said harvested MSCs or progeny thereof for at least 10 population doublings to obtain culture-expanded MSCs.

42. The method according to claim 41, wherein collagenase concentration is 0.05% wt/vol.

43. The method according to claim 41, wherein the calcium is 330 mg/L

44. The method according to claim 41, wherein said conditions comprise mixing on an orbital rotor.

45. The method according to claim 41, wherein the seeding density is between about 2,000 and about 15,000 cells per cm² of tissue culture flask.

46. The method according to claim 41, wherein said tissue culture comprises culturing said cells on microcarrier beads or macrocarrier discs.

47. The method according to claim 41, wherein serum-supplemented media comprises 10% FCS or 5 to 10% platelet lysate.

48. The method according to claim 41, wherein prior to the cells having undergone three passages, an aliquot of said cells is cultured in a culture medium comprising allogeneic serum to identify cells suitable for continued use in the production of large scale numbers of cultured MSCs.

49. The method according to claim 48, wherein the method further comprises continued passaging, in media comprising FCS or platelet lysate, of cells identified as suitable for continued use in the production of large scale numbers of cultured MSCs.

50. A method for treating an inflammatory condition, or for alleviating pain associated with an inflammatory condition, or for treating neuropathic pain, in a subject, the method comprising topical administration to said subject of a therapeutically effective amount of a composition comprising conditioned media from in vitro culture of MSCs.

51. The method according to claim 50, wherein the MSCs are human adipose-derived MSCs.

52. The method according to claim 50, wherein the inflammatory condition is selected from the group consisting of bursitis, tendonitis, golfers wrist, tennis elbow, Achilles tendonitis, calf tear, and chilblains.

53. The method according to claim 50, wherein the in vitro culture of MSCs comprises culturing in a medium comprising platelet lysate.

54. The method according to claim 53, wherein composition comprises high molecular mass glycoconjugate-enriched conditioned media.

55. The method according to claim 50, wherein the composition comprising conditioned media further comprises culture-expanded MSCs.

56. The method according to claim 55, wherein the culture expanded MSCs are multiply-passaged MSCs.

57. A method for preparing a composition comprising stable secretome, wherein said stability comprises retention of at least 60% activity after one month at room temperature, the method comprising culturing MSCs in media comprising platelet lysate for a time sufficient to permit secretion of secretome into said culture medium.

58. The method according to claim 57, wherein the stable secretome comprises one or more factors selected from the group consisting of IFN-γ, IL-8, IL-9, IL-12, IL-15, TNF-α, IL-10, MCP-1, RANTES, GM-CSF, IP-10, PDGF-bb, VEGF, IL-6, and VEGF.
